(19) United States
(12) Patent Application Publication
(10) Pub. No.: US 2003/0008819 A1
(43) Pub. Date: Jan. 9, 2003

(54) Targeting endothelium for tissue-specific delivery of agents

(76) Inventor: Jan E. Schnitzer, Encintas, CA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 10/056,230

(22) Filed: Jan. 24, 2002

Related U.S. Application Data
(63) Continuation-in-part of application No. 09/734,490, filed on Dec. 11, 2000.
Application No. 09/734,490 is a continuation of application No. 09/029,459, filed on Jun. 25, 1998, now Pat. No. 6,255,457.
Application No. 09/029,459 is a 371 of international application No. PCT/US96/14177, filed on Sep. 5, 1996.
(60) Provisional application No. 60/018,791, filed on May 31, 1996.
(60) Provisional application No. 60/018,301, filed on May 24, 1996.
(60) Provisional application No. 60/003,453, filed on Sep. 8, 1995.

(51) Int. Cl. A61K 38/17

(52) U.S. Cl. 514/12

(57) ABSTRACT

Mechanisms and routes by which molecules can be delivered into cells, particularly endothelial cells, through the action of caveolae, G domains and other plasma membrane domains and components, are described, as are delivery systems comprising antibodies to caveolar proteins or receptors and also agents conjugated to the antibody or ligand, which can be used to deliver agents to, into, and/or across the endothelium.

### RELATED APPLICATIONS

This application is a Continuation-in-part which claims priority to U.S. application Ser. No. 09/734,490, filed on Dec. 11, 2000, which is a Continuation of U.S. application Ser. No. 09/029,459, filed Jun. 25, 1998, which is the U.S. National stage of International Application No. PCT/US96/14177, filed on Sep. 5, 1996, published in English, which claims the benefit of U.S. Provisional Application No. 60/018,791, filed May 31, 1996, which claims the benefit of U.S. Provisional Application No. 60/018,301, filed May 24, 1996, and which claims priority to U.S. application Ser. No. 08/582,917, filed Jan. 4, 1996, now U.S. Pat. No. 5,776,770, which claims the benefit of U.S. Provisional Application No. 60/003,453, filed Sep. 8, 1995. The teachings of these applications are incorporated by reference in their entirety.

### GOVERNMENT SUPPORT

Work described herein was supported in part by grants HL43278, HL52766, HL58216 and IA33372 from the National Institutes of Health. The U.S. Government has certain rights in the invention.

### BACKGROUND OF THE INVENTION

Molecular medicine has discovered many new therapeutic modalities using state-of-the art techniques in molecular biology. High through-put, in vitro assays that screen for pharmacological actions on the desired cell type are frequently used to design new drugs. Although such agents are certainly justified by their success in vitro, they frequently perform much less effectively in vivo where the agent must reach its target cells in a tissue in sufficient quantities to be potent while sparing bystander organs (Jain, R. K., Nat Med 4:655-7 (1998)). Depending on the route of administration, the endothelium and/or epithelium form significant barriers that greatly limit the in vivo accessibility of many drugs, antibodies, and gene vectors to their intended target sites of pharmacological action, namely the cells inside the tissue ((Jain, R. K., Nat Med 4:655-7 (1998); Miller, N. and Vile, R., FASEB J. 9:190-199 (1995); Thrush, G. R. et al., Ann. Rev. Immunol. 14:49-71 (1996); Tomlinson, E., Advanced Drug Delivery Reviews 1:87-198 (1987)). For example, poor tissue penetration has hindered many monoclonal antibodies from reaching their cell-specific antigens to achieve effective tissue- or cell-directed pharmaco-delivery in vivo (Jain, R. K., Nat Med 4:655-7 (1998); Thrush, G. R. et al., Ann. Rev. Immunol. 14:49-71 (1996); Tomlinson, E., Advanced Drug Delivery Reviews 1:87-198 (1987); Dvorak, H. F. et al., Cancer Cells 3:77-85 (1991); Weinstein, J. N. and van Osdol, W., Int. J. Immunopharmacol. 14:457-463 (1992)). The microvascular endothelium in most organs acts as a significant barrier to the free passage of blood-borne molecules and cells to the underlying interstitium and tissue cells (Schnitzer, J. E., Trends in Cardiovasc. Med. 3:124-130 (1993); Renkin, E. M., J. Appl. Physiol. 134:375-382 (1985)). Specific transport mechanisms are expected to exist for the transendothelial transport of essential circulating blood macromolecules to the subendothelial space in order to meet the metabolic needs of the surrounding tissue cells (Schnitzer, J. E., Trends in Cardiovasc. Med. 3:124-130 (1993)).

Continuous endothelium contain distinct flask-shaped invaginations in the plasma membrane called caveolae that are open to the luminal blood vessel space where circulating molecules may enter them (Schneeberger, E. E. and Hamelin, M., Am. J. Physiol. 247:H206-H217 (1984); Milici, A. J. et al., J. Cell Biol. 105:2603-2612 (1987); Ghitescu, L. et al., J. Cell Biol. 102:1304-1311 (1986)). These caveolae may provide a trafficking pathway for macromolecules into and possibly across cells (Schnitzer, J. E., N. Engl. J. Med. 339:472-4 (1998); Schnitzer, J. E., Trends in Cardiovasc. Med. 3:124-130 (1993); Renkin, E. M., J. Appl. Physiol. 134:375-382 (1985); Schneeberger, E. E. and Hamelin, M., Am. J. Physiol. 247:H206-H217 (1984); Milici, A. J. et al., J. Cell Biol. 105:2603-2612(1987); Ghitescu, L. et al., J. Cell Biol. 102:1304-1311 (1986)). Some investigators have concluded that caveolae are not dynamic but rather static structures based on morphological studies showing few plasmalemmal vesicles existing free and unattached to other membranes inside the cell (Severs, N. J., J. Cell Sci. 90:341-8 (1988); Rippe, B. and Haraldsson, B., Acta Physiol. Scand. 131:411-428 (1987); Bundgaard, M. et al., Proc. Natl. Acad. Sci. USA 76:6439-6442 (1979); Bundgaard, M., Federation Proc. 42:2425-2430 (1983)). Yet, caveolae can bud from the plasma membrane via a dynamin-mediated, GTP-dependent fission process (Oh, P. et al., J. Cell Biol. 141:101-114 (1998); Schnitzer, J. E. et al., Science 274:239-242 (1996)) and contain key functional docking and fusion proteins (Schnitzer, J. E. et al., Science 274:239-242 (1996); McIntosh, D. P. and Schnitzer, J. E., Am. J. Physiol. 277:H2222-2232 (1999); Schnitzer, J. E. et al., Science 269:1435-1439 (1995); Schnitzer, J. E. et al., J. Biol. Chem. 270:14399-14404 (1995); Schnitzer, J. E. et al., Am. J. Physiol. 37:H48-H55 (1995)). Whether caveolae can traffic their cargo across cells (transcytosis) has previously been unproven, primarily because comparative analysis has not been possible using probes capable of targeting caveolae with high affinity and specificity in vivo vs. physically identical, nontargeting control probes. The utility of caveolae in overcoming cell barriers to facilitate efficient pharmacodelivery in vivo has previously been unknown.

### SUMMARY OF THE INVENTION

The present invention is derived from methods of isolating and purifying microdomains or components of the cell surface or plasma membrane; from the resulting purified microdomains and components (e.g., proteins, peptides, lipids, glycolipids); from antibodies to the purified microdomains and components; and uses therefor. Described herein are methods of purifying microdomains of plasma membranes, including caveolae, microdomains of GPI-anchored proteins (G-domains) and membrane fragments consisting essentially of caveolae and G domains, as well as the resulting purified microdomains and uses therefor. Also described herein are methods of purifying detergent-sensitive (detergent-soluble) microdomains and cytoskeletal components, as well as the resulting purified microdomains and uses for these components.

Plasma membrane components purified by methods of the present invention are useful, directly or indirectly, in the transport of molecules, such as drugs, DNA molecules, or antibodies in various cells (e.g., epithelial, endothelial, fat cells). For example, such agents targeted to caveolae in endothelium will be transported by the caveolae into and/or across the endothelium, and, thus, are useful in breaking through a critical barrier which prevents entry of many molecules, including drugs, into most tissues from the circulating blood.

Caveolae and other plasma membrane components identified as described herein can be used to identify mechanisms or routes by which molecules can be delivered into cells, particularly endothelial cells, through the action of caveolae, G domains and other plasma membrane domains and components. For example, in one embodiment, molecules residing in caveolae can be targeted by antibodies or natural ligands to caveolar proteins or receptors, thereby bringing agents conjugated to the antibody or ligand to, into, and/or across the endothelium. Representative agents which can be conjugated to the antibody or ligand include, for example, a drug, including a peptide or small organic molecule; a gene encoding a therapeutic or diagnostic peptide/protein; or another antibody. The antibodies or ligands can be introduced into an individual, in whom they act to deliver the agent. Alternatively, in another embodiment, purified caveolae can be modified to serve as drug delivery vehicles, such as by introducing into them an agent, such as a drug, including a peptide or small organic molecule; a gene encoding a therapeutic or diagnostic peptide/protein; or an antibody. The resulting modified purified caveolae can be introduced into an individual, in whom they act to deliver the agent.

Thus, purified caveolae, G domains, and co-isolated caveolae and G domains as described herein are useful for the identification of molecules and proteins which are involved in intra- or trans-cellular transport and cell surface signal transduction and communication. They thus make it possible to identify new means by which molecules can be delivered to plasma membranes and, if desired, enter the cell, cross from one side of the cell to the other, or provide a signal to the cell that alters its function. For example, the purified caveolae and the purified G domains can be used to make specific probes or antibodies. Antibodies or ligands which are specific to the caveolae, or to the purified G domains, can be used as vectors to target the caveolae or G domains and to influence the transport of molecules into and/or across the plasma membrane. Such vectors can be used to deliver agents into and/or across the cell, such as drugs, genes, or antibodies, and particularly to deliver agents into and/or across the endothelium. The vectors can contain an active component (e.g., the drug, gene, antibody, or other agent) and a transport component (e.g., an antibody or ligand specific to caveolae or to a protein, peptide or ligand within caveolae).

In addition, the purified caveolae and the G domains of the current invention can be used to deliver agents into and/or across the cell, such as drugs, genes, or antibodies and particularly to deliver agents into and/or across the endothelium. These domains can also be used as transfer vehicles. For example, lipid-anchored molecules added to or naturally found in the purified caveolae or purified G domains can, upon introduction into the peripheral blood circulation, interact with blood vessel endothelium, and be transferred to that endothelium, including directly into the plasma membrane.

### BRIEF DESCRIPTION OF THE DRAWINGS

FIG. 1 is a schematic representation of isolation of highly purified plasma membrane caveolae.

FIG. 2 is a schematic representation of isolation of GPI-anchored protein microdomains from plasma membranes.

FIG. 3 is a schematic representation of isolation of caveolae associated with GPI-anchored protein microdomains.

FIG. 4 is a graphic representation of the percent distribution of specific proteins in plasma membrane subfractions.

### DETAILED DESCRIPTION OF THE NVENTION

The present invention is derived from methods of purifying plasma membrane microdomains and components; methods of producing the purified plasma membrane microdomains and components; antibodies that are specific for the purified plasma membrane microdomains and components; and uses for these purified plasma membrane microdomains and components, including identifying molecules involved in intra- or trans-cellular transport or cell surface signal transduction and communication and targeting of the endothelium (e.g., for delivery of an agent or for gene therapy). A description of the purification methods is set forth herein, followed by a description of uses of the purified components.

The methods of purifying and/or producing plasma microdomains and components described herein can be carried out on any cell type (e.g., endothelial, epithelial, and fat cells) whose plasma membrane contains the desired component. Components which can be isolated by the present method include caveolae, G domains, membrane fragments consisting essentially of caveolae associated with G domains, detergent soluble components, and cytoskeletal components.

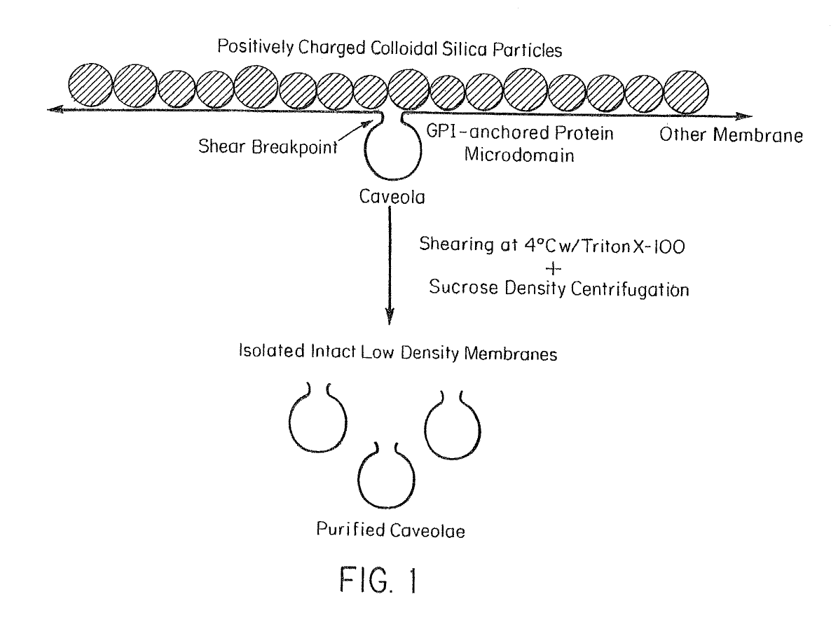

In one embodiment, the methods relate to caveolae purified from plasma membranes, such as endothelial cell plasma membranes, by the method described herein. As described in detail in Examples 1 and 2 and represented schematically in FIG. 1, highly purified caveolae are obtained from isolated luminal endothelial cell plasma membranes. The caveolae of the invention are purified based on both morphological and biochemical criteria. They are substantially free of microdomains of GPI-anchored proteins, GPI-anchored proteins and Rab5 (a guanosine triphosphate (GTP)-binding protein that is found in detergent-resistant complexes, as described below). By electron microscopy, this fraction contains a rather homogeneous population of vesicles predominantly <1000 Å in diameter, and have a morphologically distinctive appearance of caveolae (Schnitzer, J. E., et al., Proc. Natl. Acad. Sci. USA 92:1759 (1995)). Purified caveolae have been obtained by coating the surface of cells (such as endothelial cells) with cationic colloidal silica particles; separating the silica-coated cell plasma membranes from the remainder of the cell and any associated tissue, to produce silica-coated cell plasma membranes; stripping the caveolae (present on the side of the membrane opposite that to which the silica particles attached) from the membrane by a membrane disruption technique (such as shearing or sonication); and separating the caveolae from the other plasma membrane components (which include the remaining silica-coated plasma membranes rich in GPI-anchored proteins but devoid of caveolae and caveolin). This separation is carried out on the basis of density, such as by sucrose density gel centrifugation. In one embodiment, endothelial cell plasma membranes are subjected to shearing in the presence of a detergent (e.g., Triton X-100) during homogenization at an appropriate temperature (e.g., approximately 4° C.-8° C.). Low temperature is necessary if detergent is used, because caveolae are only detergent-resistant at low temperatures. At physiologic temperature (37° C.), caveolae are solubilized by detergent. Detergent appears to facilitate the removal of caveolae from their attachment point on the plasma membrane, and thus facilitate the stripping process, but is not essential or necessary for the process. In a second embodiment, endothelial cell plasma membranes are subjected to shearing during homogenization in the absence of detergent.

In either embodiment, the result is separation of caveolae from other cell membrane components and isolation of purified caveolae. Characterization of the purified caveolae showed that they are very enriched in caveolin; the glycolipid GM₁; the plasmalemmal Ca²⁺-dependent adenosine triphosphatase; and the inositol 1,4,5-triphosphate receptor. These four molecules have all been shown by independent means (localization by electron microscopy) to reside on the cell surface almost exclusively in caveolae (Dupree, P., et al., EMBO J. 12:1597 (1993); Parton, R. G., J. Histochem. Cytochem. 42:155 (1994); Rothberg, K. G., et al., Cell 68:673 (1992); Montessano, R., et al., Nature 296:651 (1982); Fujimoto, T., et al., J. Cell. Biol. 119:1507 (1992); Fujimoto, T., J. Cell. Biol. 120:1147 (1993)) and thus represent key markers of the caveolae. These four caveolar markers which are all present amply in the silica-coated plasma membrane pellet, fractionate almost totally into the purified caveolae. Little if any remains in the other membrane fractions. In contrast, angiotensin-converting enzyme, band 4.1 and β-actin, which were all present amply in the silica-coated plasma membrane fraction (P) are almost totally excluded from the purified caveolae.

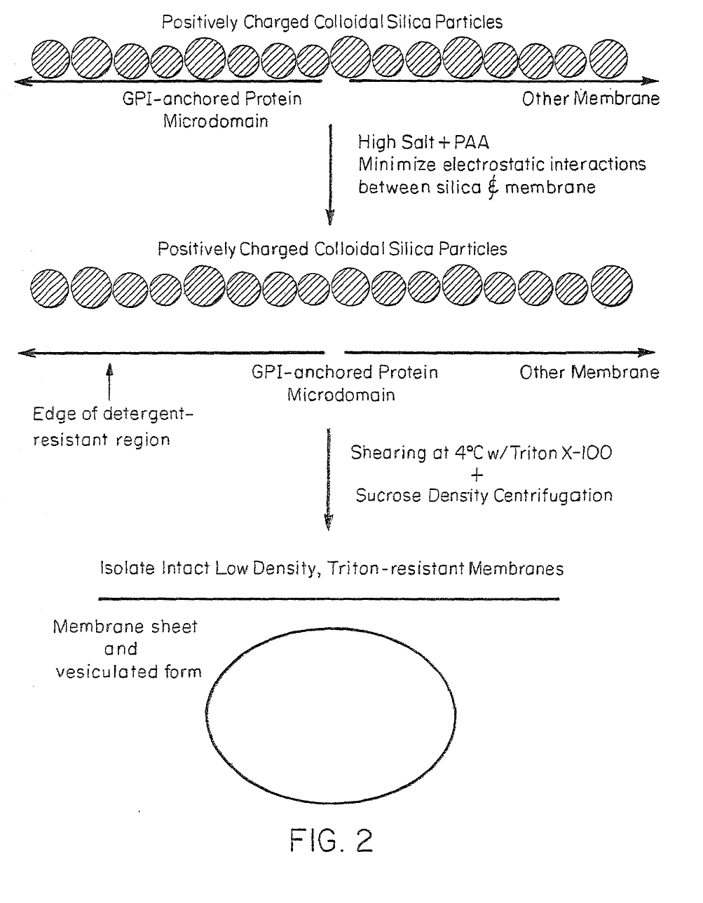

In a further embodiment, the methods relate to microdomains of GPI-anchored proteins (G domains) purified from plasma membranes, such as endothelial cell plasma membranes. The microdomains of GPI-anchored proteins are purified, in that they are substantially free of caveolae, caveolin and GM₁ (a lipid-anchored, cholera toxin-binding ganglioside that has been localized with gold labeling inside the caveolae, as described below). As described in Example 3 and represented schematically in FIG. 2, G domains were isolated from cell plasma membranes (e.g., endothelial plasma membranes) originally isolated from the cells (e.g., by use of silica coating, as described in Example 1) and stripped of caveolae. The isolated silica-coated plasma membranes stripped of caveolae were subjected to a salt concentration sufficiently high to reduce/minimize electrostatic interactions between the silica particles and plasma membrane, resulting in separation of the plasma membranes from the particles. The resulting non-coated plasma membranes (previously stripped of caveolae) were subjected to a membrane disruption technique (e.g., shearing) in the presence of a detergent (e.g., Triton X-100) and then subjected to a separation technique which separates components based on density (e.g., sucrose density centrifugation), resulting in isolation of intact G domains, which are low density, detergent-insoluble (resistant) membrane microdomains; are rich in GPI-anchored proteins; and are substantially free of caveolae.

The data herein indicate that GPI-anchored proteins partition into diffusion-restrictive microdomains, some of which may associate with caveolae as an annular region at the opening or neck of the caveolae. Because both caveolae and G domains are resistant to detergent solubilization, the normally flat membrane region surrounding the opening of the caveolea has been excised from the plasmalemma to form an intact large vesicle with a caveolea still attached and located usually inside but sometimes outside of the vesicle upon detergent extraction. The silica coating prevents the co-isolation of this microdomain with the caveolae, and allows separate isolation of caveolae and the G domain.

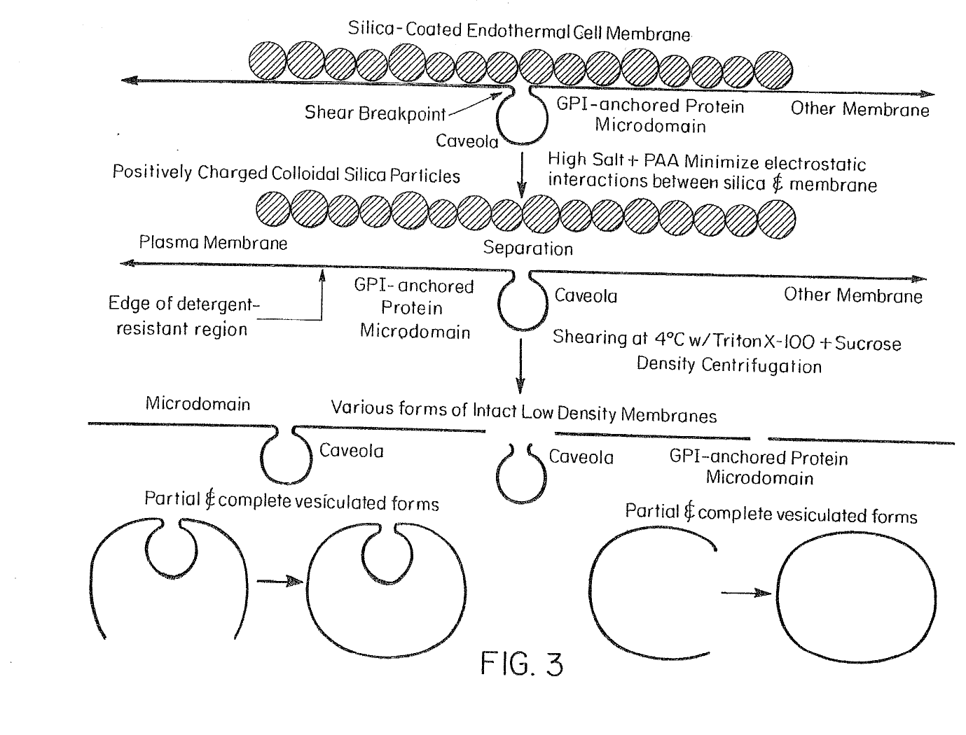

In another embodiment, the present invention relates to plasma membrane domains which consist essentially of caveolae, G domains (some of which are associated with each other) and are purified, for example, from endothelial cell plasma membranes. The term, “associated with,” as used herein, indicates that some of the caveolae are attached to the G domains, rather than being separated. As described in Example 4, and represented schematically in FIG. 3, plasma membrane domains consisting essentially of caveolae, G domains, and caveolae associated with G domains are produced by isolating silica-coated cell membranes with caveolae still attached, as described above; subjecting the silica-coated cell plasma membranes to high salt to separate the silica coating from the membranes, and subjecting the membranes to a membrane disruption technique, such as shearing or sonication, in the presence of an appropriate detergent, such as Triton X-100. This results in separation of the membrane into various components, including caveolae associated with G domains. Domains consisting essentially of caveolae, G domains, together with caveolae associated with G domains are detergent resistant, and can be separated from other components on the basis of density, such as by sucrose density centrifugation.

In another embodiment, other components of the cell plasma membrane can be isolated. Such components include detergent-soluble components or cytoskeletal components which remain after isolation of caveolae, G domains, or caveolae associated with G domains, as described above. These other components are substantially free of caveolae and/or G domains. For example, after isolation of G domains that are substantially free of caveolae, as described above, the remaining detergent-soluble components can be isolated and purified.

As a result of these discoveries, methods are now available to isolate caveolae that are substantially free of microdomains of GPI-anchored proteins and other cell components; G domains that are substantially free of caveolae and other cell components; and co-isolated plasma membrane microdomains that consist essentially of caveolae, G domains and caveolae associated with G domains. The caveolae, G domains, and/or co-isolated plasma membrane microdomains can be isolated from any endothelial cell plasma membrane from any tissue. Tissues from which endothelial cell membrane can be used include vascular, pulmonary, cardiac, cerebral, nephric, hepatic and endocrinous tissue, including the vascular system, lung, heart, liver, kidney, brain and other organs. For example, caveolae, G domains and/or co-isolated plasma membrane domains can be isolated from vascular endothelium by perfusion through the blood vessels, or intestinal epithelium by perfusion through the intestine. In addition, caveolae, G domains and/or co-isolated plasma membrane domains can also be isolated from a variety of cells, such as those grown in cultures.

In a specific embodiment of the invention, specific microdomains are isolated from endothelial cell plasma membranes by first isolating cell membranes, by forming a coating of an adherent, first ionic material on a luminal surface of the endothelial cell membrane by perfusing the ionic material into a luminal cavity adjacent to the endothelial cell membrane; crosslinking the coating to form a pellicle adherent to the endothelial membrane (referred to as a pellicle-endothelial membrane complex) by contacting the luminal surface of the ionic material coating with an oppositely charged ionic material reactive with the first ionic material; and separating the complex from other tissue elements by a method based on differences in size or density (e.g., by centrifugation), thus producing coated membrane pellets. Subsequently, specific microdomains can be isolated. For example, caveolae can be isolated by stripping caveolae from the coated membranes by shearing during homogenization, in the presence or absence of detergent; and isolating the caveolae from other components on the basis of density, such as by sucrose density gradient centrifugation. Caveolae isolated by this method are substantially free of G domains. Alternatively, G domains can be isolated by isolating the coated membranes after isolating and removing the caveolae; subjecting the coated membranes to high salt to remove the silica coating; and isolating the membranes. These membranes isolated by this method consist essentially of G domains.

In the preferred embodiments of the invention, the first ionic material is colloidal silica and the second ionic material is an acrylic polymer. One of many alternatives is to use magnetic particles to coat the membranes, which can subsequently be isolated, using standard magnetic techniques.

The purified caveolae, the purified microdomains of GPI-anchored proteins, and the purified co-isolated plasma microdomains comprising caveolae associated with G domains are useful for the identification of molecules and proteins which are involved in intra- or trans-cellular transport. Furthermore, the caveolae and the G domains are purified and, thus, can be used to distinguish and identify proteins which are limited to either the caveolae or the microdomains, but are not present in both. For example, purified caveolae can be used to generate antibodies, either monoclonal or polyclonal, using standard techniques. The term “antibody”, as used herein, encompasses both polyclonal and monoclonal antibodies, as well as mixtures of more than one antibody reactive with caveolae (e.g., a cocktail of different types of monoclonal antibodies reactive with the caveolae). The term antibody is further intended to encompass whole antibodies and/or biologically functional fragments thereof, chimeric antibodies comprising portions from more than one species, humanized antibodies and bifunctional antibodies. Biologically functional antibody fragments which can be used are those fragments sufficient for binding of the antibody fragment to purified caveolae. Once the antibodies are raised, they are assessed for the ability to bind to purified caveolae. Conventional methods can be used to perform this assessment.

The chimeric antibodies can comprise portions derived from two different species (e.g., a constant region from one species and variable or binding regions from another species). The portions derived from two different species can be joined together chemically by conventional techniques or can be prepared as single contiguous proteins using genetic engineering techniques. DNA encoding the proteins of both the light chain and heavy chain portions of the chimeric antibody can be expressed as contiguous proteins.

Monoclonal antibodies (mAb) reactive with purified caveolae can be produced using a variety of techniques, such as somatic cell hybridization techniques (Kohler and Milstein, Nature 256: 495-497 (1975)), in situ techniques and phage library methods. For example, purified caveolae can be used as the immunogen. Alternatively, synthetic peptides corresponding to portions of proteins found in the caveolae can be used as immunogens. An animal is immunized with such an immunogen to obtain antibody-producing spleen cells. The species of animal immunized will vary depending on the specificity of mAb desired. The antibody producing cell is fused with an immortalizing cell (e.g., a myeloma cell) to create a hybridoma capable of secreting antibodies. The unfused residual antibody-producing cells and immortalizing cells are eliminated. Hybridomas producing desired antibodies are selected using conventional techniques and the selected hybridomas are cloned and cultured.

Polyclonal antibodies can be prepared by immunizing an animal in a similar fashion as described above for the production of monoclonal antibodies. The animal is maintained under conditions whereby antibodies reactive with purified caveolae are produced. Blood is collected from the animal upon reaching a desired titer of antibodies. The serum containing the polyclonal antibodies (antisera) is separated from the other blood components. The polyclonal antibody-containing serum can optionally be further separated into fractions of particular types of antibodies (e.g., IgG, IgM).

Alternatively, purified G domains or purified silica-coated membranes can also be used to generate antibodies, as can any membrane fraction obtained as described herein. Synthetic peptides corresponding to portions of proteins from any of the fractions can also be used. Purified caveolae can be used to identify those antibodies which bind caveolae. Alternatively, purified G domains can be used to identify those antibodies which bind G domains.

Antibodies as described above can be used to identify further the proteins associated with intra- and trans-cellular transport: for example, the antibodies can be applied to endothelium in order to determine whether they interfere with transport in endothelium. Antibodies can additionally be used as vectors to deliver agents into and/or across the endothelium. For example, as described in Examples 8 and 9 below, monoclonal antibodies have been generated which recognize antigens found primarily in purified caveolae, and which can be used for tissue-specific transcytosis in vivo. Most of the antibodies recognize endothelia; a few are specific for continuous endothelia. Furthermore, antibodies can be generated which are tissue specific. Two of the antibodies described in Example 8 recognize lung tissue. Tissue-specific antibodies can be used as transport agents to deliver agents, such as antibodies, drugs, genes, diagnostic agents, or other molecules to a specific tissue, and particularly to the caveolae of a specific tissue, so that the agents can be delivered to and/or across the endothelium.

The purified caveolae or G domains of the current invention can also be used to target the endothelium, such as for delivery of an agent or for gene therapy. Agents which target caveolae or the G domains may be more easily delivered to the cell and, if desired, enter the cell, cross from one side of the cell to the other, or provide a signal to the cell that alters its function. For example, agents such as antibodies, drugs, or other molecules which bind to G domains or to proteins in caveolae (e.g., the insulin receptors) target the caveolae or the G domains and may thereby be moved into and/or across the epithelium. Such antibodies, drugs, or other molecules can also be used as transport agents, by conjugating another agent (such as a drug or a gene) to the agent which targets the caveolae or G domain. In this manner, the purified caveolae and the purified microdomains of GPI-anchored proteins are also useful as transport vehicles, to move agents across the endothelial layer. The physical association of G domains with caveolae suggests functional interplay between them; therefore, these structures may provide a platform for ligand processing by integrating signal transduction with membrane transport. Binding of natural ligands or antibodies to GPI-linked proteins can induce clustering (Schroeder, R., et al., Proc. Natl. Acad. Sci. USA. 91:12130 (1994); Mayor, S., et al., Science 264:1948 (1994)), internalization by caveolae via potocytosis (Anderson, R. G. W., et al., Science 255:410 (1992); Rothberg, G., et al., J. Cell. Biol. 111:2931 (1990)) or endocytosis (Keller, E.-A., et al., EMBO J., 3:863 (1992); Bamezai, A., et al., Eur. J. Immunol. 22:15 (1992); Parton, R. G., et al., J. Cell. Biol. 127:1199 (1994)), and even cell activation (Thompson, L. F., et al., J. Immunol. 143:1815 (1989); Korty, P. E., et al., J. Immunol. 146:4092 (1991); Davies, L. S., J. Immunol. 141:2246 (1988)). Signaling may regulate caveolar processing (Parton, R. G., et al., J. Cell Biol. 127:1199(1994); Smart, E. J., et al., J. Cell. Biol. 124:307(1994)), and various mediators of signaling may reside in caveolae (Schnitzer, J. E. et al., Proc. Natl. Acad. Sci., USA 92:1759 (1995); Fujimoto, T., et al., J. Cell. Biol. 119:1507 (1992); Fujimoto, T., J. Cell. Biol. 120:1147 (1993); Schnitzer, J. E., et al., J. Biol. Chem. 270:14399 (1995)). Lastly, surface-bound molecules are endocytosed or transcytosed by caveolae in endothelium (Montessano, R., et al., Nature 296:651 (1982); Schnitzer, J. E., Trends Cardiovasc. Med. 3:124 (1993); Oh, P., et al, J. Cell Biol. 127:1217 (1994); Schnitzer and Oh, P., J. Biol. Chem. 269:6072 (1994); Millici, A. J., et al., J. Cell Biol. 105:2603 (1987); Schnitzer, J. E., et al., J. Biol. Chem. 264:24544 (1992); Schnitzer, J. E, and Bravo, J., J. Biol. Chem. 268:7562 (1993)). Disassembly of caveolae prevents such transport (Oh, P., et al., J. Cell Biol. 127:1217 (1994)), and molecular mapping of purified caveolae reveals the presence of SNARE fusion proteins and guanosine triphosphatases necessary for regulated N-ethylmaleimide-sensitive vesicular transport (Schnitzer, J. E., et al., J. Biol. Chem. 270:14399 (1995); Schnitzer, J. E., et al., Am. J. Physiol. 37:1148 (1995)). Thus, as a result of the purification of the endothelial caveolae as described herein, it has become apparent that caveolae are indeed vesicular carriers and contain the molecular machinery for integrating signaling with carrier transport. Dynamic ligand processing via clustering, signaling, and vesicular transport may occur through the association of the GPI-linked protein microdomains with caveolae or even possibly via caveolar formation. Such specialized distinct microdomains may exist separately or associated with each other not only to organize signaling molecules but also to process surface-bound ligands differentially.

Thus, caveolae (and associated membrane components, such as GPI-anchored proteins) play a key role in the transport of molecules into and across many types of cell membranes and particularly are involved in transporting molecules into and/or across the endothelium and, as a result, across the endothelial barrier. This is of considerable interest and value because of the role the endothelium plays in many tissues in the body as a barrier to passage of substances, such as drugs and other agents which can have a beneficial effect if they are made available to the underlying tissue. Work described herein makes it possible to identify means by which transport across cell membranes, particularly endothelial cell membranes, can be facilitated and, if desired, effected in a tissue specific manner (i.e., directed to a selected tissue type or types through caveolae and/or GPI-anchored proteins which are cell-type specific). In addition to mediating, controlling, and/or regulating the transport of various molecules including ions, small molecules, proteins, and even water into cells, such as endothelial cells, caveolae have a role in cell surface signal transduction and communication. Recent work has shown that caveolae may also act in interactions between cells and surrounding tissues and fluids and that in doing so, they store and process messenger molecules (e.g., CAMP, Calcium) and initiate phosphorylation cascades by using kinases such as non-receptor tyrosine kinases. (See, e.g., Anderson, R. G. W., Proc. Natl. Acad. Sci. USA, 90:10909-10913 (1993); Anderson, R. G. W., Current Opinion in Cell Biology 5:647-652 (1993)).

As a result, the availability of purified caveolae, G domains and membrane domains consisting essentially of caveolae and G domains, as well as antibodies that are specific to purified caveolae, G domains, or membrane domains consisting essentially of caveolae and G domains, makes it possible to deliver molecules, such as drugs and diagnostic agents, to and/or across cell membranes, such as endothelial cell membranes, and to alter cell signaling and communication, such as by interacting with molecules shown to be present in or associated with caveolae and/or G domains and to have a role in signal transduction and communication. For example, purified caveolae of the present invention have been characterized as to constituent proteins and other components and can be further assessed to identify components which play key roles in transport or signal transduction and communication, either in a variety of cell types (to permit a general effect on cells) or in a specific cell type (to permit a selective effect). For example, plasma membrane components can be identified which improve transport of a drug such as an immunotoxin (to be delivered to a tumor or other malignancy) for cancer therapy or a selective stimulant (to be delivered to heart tissue and, more specifically cross the heart endothelial barrier to the underlying and normally less accessible cardiomyocytes) for treatment of cardiac conditions. Alternatively, plasma membrane components can be identified which will facilitate gene delivery into cells, such as epithelial cells, in which the gene will be processed to produce a therapeutic or diagnostic protein or peptide or an antisense nucleic acid. For example, if the goal is to introduce a gene into blood vessels in order to produce and secrete an anticoagulant or blood thinning protein, the appropriate target will be the endothelium, especially in heart tissue. Caveolae and/or GPI-anchored proteins present in (and possibly unique to) endothelial cell plasma membranes in heart and/or blood vessels can be identified, using purified caveolae and/or G domains described herein; and used to target or direct a gene delivery vehicle (such as a plasmid or viral vector, or protein- or peptide-DNA conjugate) to heart tissue and/or blood vessels. Alternatively, antibodies to caveolae and/or GPI-anchored proteins present in or unique to specific tissues can be used to target or direct a gene delivery vehicle to the specific tissue. Similarly, lipid-anchored proteins found in caveolae or G domains can be introduced into the peripheral blood circulation, where they interact with the epithelium of the blood vessels and can be transferred to the blood vessel epithelium.

Furthermore, the endothelium is responsive to multiple physical and chemical factors in its local tissue microenvironment and plays a primary or secondary role in many vascular and extravascular diseases such as cancer, atherosclerosis, diabetic microangiopathies, and cardiac ischemia (Folkman, J., Nature Medicine 1:27-30 (1995)). Thus, proteins present in tumor blood vessels can be targeted in directed therapy, by direct delivery of an agent that targets the tumor endothelium for selective destruction, while avoiding bystander noncancerous tissues.

Directing delivery of a drug or other agent which is to enter a cell through the action of caveolae, GPI-anchored proteins or other plasma membrane domain can be carried out by using as a “probe” or transporting molecule a molecule (such as an antibody, a peptide, a virus, a ligand) which has a relatively high affinity interaction with a component of caveolae, G domains or other plasma membrane domain. The probe or transporting molecule can itself be the drug or agent whose entry into cells, such as endothelial cells, is desired or can be attached to a second molecule whose entry into cells is desired. The transporting molecule and the attached molecule will be extracted from the blood and accumulated in the targeted tissue by action of the caveolae. For example, an antibody or other molecule which recognizes a protein found only in lung caveolae, such as the antibodies described in Example 8, can be used to direct a drug, which can be the antibody or other molecule, or can be delivered by their action, to lung tissue for therapeutic or diagnostic purposes. The agent will be accumulated in the lung tissue via lung caveolae and, thus, made available to the tissue for the desired effect. Similarly, such probes or transporting molecules (which target caveolae in a specific tissue type or generally bind caveolae on many tissue types) can be used to introduce drugs or other agents into a variety of tissue types.

Proteins and other components of caveolae or G domains which can be targets for the probes or transporting molecules can be identified using purified caveolae or G domains of the present invention. Conversely, probes or transporting molecules can also be identified using purified caveolae or G domains described herein. To identify such molecules, standard assays can be used, including: two-dimensional gel analysis followed by microsequencing, Western blotting with antibodies to known proteins (as described herein), or blotting with antibodies as described above.

It is also possible to use purified caveolae, G domains and membrane fragments consisting essentially of caveolae and G domains as delivery vehicles. For example, purified caveolae can be modified to contain a drug or other agent (such as a chemotherapeutic) to be delivered to a tissue of interest. The modified purified caveolae are introduced into an individual in need of the agent by an appropriate route such as intravenously, intramuscularly, topically, or by inhalation spray. For example, modified caveolae containing an agent can be administered into the lungs of an individual in need of a chemotherapeutic agent for lung cancer by an aerosol or inhalation spray. In the lung tissue, the caveolae act as delivery vehicles and the agent is delivered to the affected cells.

The invention is further illustrated by the following Examples.

### EXAMPLES

When cells are isolated from tissues and grown in culture, there can be a substantial loss of caveolae from the cell surface, especially for endothelial cells (J. E. Schnitzer et al., Biochem. Biophys. Res. Commun. 199:11 (1994). Such losses (often >100-fold) represent a substantial alteration in plasma membrane organization and may reflect a major perturbation in caveolar function and even GPI-linked protein clustering. Thus, the relationship between GPI-anchored proteins and caveolae was explored under conditions to avoid potential influences of cell culture, and also to avoid antibody effectors and contamination from intracellular compartments.

All of the membrane subfractions described herein were isolated after exposure to detergent in order to be consistent and limit the number of variables in the comparison of the subfractions. However, caveolae can be sheared and isolated from silica-coated membrane pellets without detergent, although less efficiently. As noted in (Kurzhalia, T. V., et al., Trends Cell Biol. 5:187-189 (1995)), the usual protocol was followed for caveolae isolation, with the exception that Triton X-100 was omitted and, for shearing purposes, the number of homogenization strokes was increased to 48 or 60 from 12.

The following methods and materials were used to selectively isolate the luminal endothelial plasmalemma with its subtending caveolae from rat lung microvasculature and to purify caveolae from the plasmalemmal fraction.

Methods

Antibodies.

Mouse monoclonal antibody to caveolin was from Zymed or Transduction Laboratories (Lexington, Ky.); rabbit polyclonal antibody to angiotensin converting enzyme (ACE) was from R. Skidgel (University of Illinois); rabbit polyclonal antibody to band 4.1, from V. Marchesi (Yale University); mouse monoclonal antibody to Ca²⁺-ATPase, from Affinity BioReagents (Neshanic Station, N.J.); mouse monoclonal antibody to β-actin, from Sigma; and goat polyclonal antibody to IP₃ receptor, from Solomon H. Snyder and Alan Sharp (Johns Hopkins University). Sources for other reagents were as before (Schnitzer, J. E., et al., J. Cell Biol. 127:1217 (1994); Schnitzer, J. E. and Bravo, J., J. Biol. Chem. 268:7562 (1993); Schnitzer, J. E. and Oh, P., J. Biol. Chem. 269:6072 (1994); and Jacobson, B. S., et al., Eur. J. Cell Biol. 58:296 (1992)).

In situ Perfusion of Rat Lungs for Silica Coating of the Luminal Endothelial Cell Surface.

The lungs of anesthetized male Sprague-Dawley rats were ventilated after tracheotomy and then perfused as described (Schnitzer, J. E. and Oh, P., J. Biol. Chem. 269:2072 (1994); Jacobson, B. S., et al., Biochem. Biophys. Res. Commun. 199:11 (1994)). In brief, the right cardiac ventricle was injected with 0.5 ml of Ringer's solution at pH 7.4 (111 mM NaCl/2.4 mM KCl/1 mM MgSO₄/5.5 mM glucose/5 mM Hepes/0.195 mM NaHCO₃) containing 30 μM nitroprusside and 175 units of heparin before cannulation of the pulmonary artery. The lungs were perfused at 8-10 mmHg (1 mmHg=133 Pa) with the following solutions (all at 10-12° C. except as noted) in order: (i) oxygenated Ringer's solution containing 30 μM nitroprusside for 90 sec at room temperature and then for 3.5 min at 10-12° C.; (ii) MBS (125 mM NaCl/20 mM Mes, pH 6.0) for 90 sec; (iii) 1% colloidal silica in MBS; (iv) MBS for 90 sec, to clear free silica from vasculature; (v) 1% sodium polyacrylate in MBS for 90 sec, to crosslink and shield membrane-bound silica; and (vi) 8-10 ml of Hepes-buffered sucrose with protease inhibitors [HBS+, pH 7, contains 0.25 M sucrose, 25 mM Hepes, leupeptin (10 μg/ml), pepstatin A (10 μg/ml), o-phenanthroline (10 μg/ml), 4-(2-aminoethyl)benzenesulfonyl fluoride (10 μg/ml), and trans-epoxysuccinyl-L-leucinamido(4-guanidono)butane (50 μg/ml)]. The lungs were excised and immersed in cold HBS+.

Purification of Luminal Endothelial Cell Membranes.

The chilled rat lungs were weighed, minced with a razor blade in a plastic dish on an aluminum block embedded in crushed ice, and then added to 20 ml of cold HBS+ for homogenization (12 strokes) in a type C Teflon pestle/glass homogenizer with a high-speed rotor run at 1800 rpm. After filtration through a 0.53-μm Nytex net followed by a 0.3-μm net, the homogenate was mixed with 102% (wt/vol) Nycodenz (Accurate Chemical and Scientific) with 20 mM KCl to make a 50% final solution and was layered over a 55-70% Nycodenz continuous gradient containing 20 mM KCl plus HBS. After centrifugation in a Beckman SW28 rotor at 15,000 rpm for 30 min at 4° C., the pellet was suspended in 1 ml of MBS and named P.

Purification of Caveolae from Silica-Coated Endothelial Cell Membranes.

Cold 10% (vol/vol) Triton X-100 was added to the suspended membrane pellet (P) as described above to make a final concentration of 1%. After nutation for 10 min at 4° C., the suspension was homogenized in a type AA Teflon pestle/glass homogenizer (10 strokes) and then brought to 40% sucrose and 20 mM KCl. A 35-0% sucrose gradient in 20 mM KCl was layered over the homogenate in a Beckman SW55 rotor tube and then centrifuged at 4° C. overnight at 30,000 rpm. A membrane layer clearly visible between 10% and 16% sucrose was collected, labeled V, and then diluted 3-fold with MBS before centrifugation for 2 hr at 13,000× g at 4° C. The resultant pellet was either processed for electron microscopy or for further analysis. In attempting to optimize the conditions for isolation of caveolae, it was found that the low-density caveolar fraction was not altered by (i) the presence of Triton X-100 throughout the sucrose gradient during centrifugation and (ii) the absence of Triton X-100 during homogenization, except that the yield of caveolae was diminished. Triton X-100 appears to facilitate the shearing of the caveolae away from the plasma membrane.

ELISA.

After the sucrose density centrifugation described above, 33 fractions of 150 μl were collected and the pellet was suspended in 150 μl of MBS (fraction 34). Aliquots of each fraction (50-100 μl) were placed in individual wells of a 96-well tray for drying overnight. After washing, the wells were blocked for 1 hr with ELISA wash buffer (EWB: 2% ovalbumin/2 mM CaCl₂/164 M NaCl/57 mM phosphate, pH 7.4), incubated for 1 hr with EWB containing antibodies (1:200) to either caveolin or ACE, washed for 1 min in EWB three times, incubated with reporter antibody conjugated to horseradish peroxidase (1:500 in EWB), and washed again. Substrate solution (50 mM Na₂HPO₄/25 mM citric acid/0.12% o-phenylenediamine dihydrochloride/0.03% H₂O₂) was added and the reaction was stopped with 4 M H₂SO₄ before the signal was read with a Molecular Devices Thermomax microplate reader.

SDS/PAGE and Immunoblotting.

As reported ((Sargiacomo, M., et al., J. Cell. Biol. 122:789 (1993); Lisanti, M. P., et al., J. Cell. Biol. 123:595 (1993); Montessano, R., et al., Nature 296:651 (1982)), the proteins of various tissue fractions were solubilized and separated by SDS/PAGE in 5-15% gels for direct analysis by silver staining or electrotransfer to nitrocellulose or poly(vinylidene difluoride) (Immobilon; Millipore) filters for immunoblotting using primary antibodies followed by appropriate ¹²⁵I-labeled reporter antibodies. Band intensities were quantified by PhosphorImager (Molecular Dynamics), densitometry of autoradiograms, and/or direct counting of γ radioactivity. Protein assays were performed with the Bio-Rad BCA kit.

### Example 1

### Isolation of Coated Membrane Pellets (P)

Isolation of caveolae associated with the endothelial cell surface has many difficulties. The endothelium represents but a small percentage of a diverse population of cells in any organ. Unfortunately, isolating endothelial cells from tissues as a primary source or even for growth in culture causes morphological changes, including a very significant loss in cell surface caveolae (Schnitzer, J. E., et al., Biochem. Biophys. Res. Commun. 199:11 (1994)). Also, noncoated vesicles that are very similar in size and density to plasmalemmal caveolae and may even contain the caveolar marker protein caveolin (Dupree, P., et al., EMBO J. 12:1597 (1993); Kurzchalia, T. V., et al., J. Cell Biol. 118:1003 (1992)), may be found in other cellular compartments such as the trans-Golgi network. Moreover, caveolae may vary according to cell type (Izumi, T., et al., J. Electron Microsc. 38:47 (1989)). To overcome the above problems, a strategy of first isolating in high yield and purity the luminal endothelial plasma membranes with associated caveolae from rat lungs in situ was used. The caveolae were then removed and isolated from this membrane fraction.

Purification of Luminal Endothelial Cell Membranes from Rat Lungs Perfused in situ

The rat lung microvasculature was perfused via the pulmonary artery with a positively charged colloidal silica solution to coat the luminal endothelial cell membrane normally exposed to the circulating blood and create a stable adherent silica pellicle that marks this specific membrane of interest (Jacobson, B. S., et al., Eur. J. Cell. Biol. 58:296 (1992)). Such a coating increased the membrane's density and was so strongly attached to the plasma membrane that after tissue homogenization, large sheets of silica-coated membrane with attached caveolae were readily isolated away from other cellular membranes and debris by centrifugation through a high-density medium (Jacobson, B. S., et al., Eur. J. Cell. Biol. 58:296 (1992)). The silica-coated membranes displayed ample enrichment for endothelial cell surface markers and little contamination from other tissue components. As shown in past work (Jacobson, B. S., et al., Eur. J. Cell. Biol. 58:296 (1992)), the typical isolated membrane sheet had caveolae attached on one side and a silica coating on the other side. By SDS/PAGE, the silica-coated membranes had a protein profile quite distinct from that of the starting lung homogenate. Moreover, quantitative immunoblotting revealed enrichments up to 30-fold in the silica-coated membrane pellets relative to the starting tissue homogenate for several proteins known to be expressed on the surface of endothelium, such as caveolin (Dupree, P., et al., EMBO J. 12:1597 (1993)) and ACE (Caldwell, P. R. B., et al., Science 191:1050 (1976)). Conversely, proteins of intracellular organelles (cytochrome oxidase and ribophorin) and even the plasma membranes of other lung tissue cells (fibroblast surface antigen) were excluded from this membrane fraction.

Detergent Resistance of Caveolin as a Marker for Caveolae

Caveolin was used as a biochemical marker for caveolae; it was found that the caveolin abundantly expressed in the silica-coated membranes was resistant to solubilization by homogenization at 4° C.-8° C. using Triton X-100 or 3-[(3-cholamidopropyl)dimethylammonio]-1-propanesulfonate but not other detergents, including octyl β-D-glucoside, SDS, deoxycholate, and Nonidet P-40. SDS/PAGE revealed that many proteins in silica-coated membranes were solubilized by Triton X-100, whereas others were not and could be sedimented by centrifugation. Immunoblotting showed that caveolin and the cytoskeletal protein band 4.1 were sedimented into the Triton-insoluble fraction, whereas ACE was found primarily in the Triton-soluble fraction.

### Example 2

### Isolation of Purified Caveolae (V)

Caveolae attached on the cytoplasmic side of the plasma membranes, opposite to the silica coating in the silica-coated membrane pellets (P), were stripped from the membranes by shearing during homogenization at 4° C. in the presence or absence of Triton X-100. They were then isolated by sucrose density gradient centrifugation to yield a homogenous population of biochemically and morphologically distinct caveolar vesicles (V). This technique is represented schematically in FIG. 1.

Isolation of Vesicles Stripped From Silica-Coated Membranes.

The silica-coated membranes (P) in Triton X-100 were stripped of protruding caveolae by shearing in a homogenizer and then subjected to sucrose density centrifugation to isolate the caveolae. Analysis of 34 fractions from the sucrose gradient revealed a peak signal for caveolin well separated from that for ACE. Little caveolin was detected in the silica-coated membrane after removal of the vesicles (P-V). Most of it was in the visible membrane band at 10-16% sucrose (fractions 6-10), which was collected, labeled V for vesicles, and examined by electron microscopy, SDS/PAGE, and immunoblotting.

Characterization of Isolated Vesicles as Caveolae.

Electron microscopy was performed (as in Dvorak, A. M., J. Electron Microsc. Tech. 6:255 (1987)) on the three main membrane fractions: original silica-coated membrane (P), isolated vesicles (V), and the silica-coated membrane pellet after removal of vesicles (P-V). In P, small, membrane-bound electron-lucent openings, or fenestrae, were visible in many vesicles in favorable sections and clearly were not part of the ostia of the caveolae directly at the cell membrane surface. These fenestrae, as described many years ago (Palade, G. E. and Bruns, R. R., J. Cell Biol. 37:633 (1968)), are characteristic of endothelial caveolae and probably indicate a previous attachment to another vesicle as part of a chain of vesicles. The P-V fraction contained silica-coated membranes without attached caveolae. The V fraction contained a rather uniform distribution of small noncoated vesicles, primarily with diameters of 50-100 nm. Higher magnification revealed vesicular structures typical for caveolae in vivo. Single plasmalemmal vesicles and chains of membrane-bound vesicles were present. The fenestrae distinctive for caveolae were easily visible in many of the vesicles. Some vesicles still maintained their narrowed necks as in caveolae that are attached to the plasmalemma of endothelium in vivo. Higher magnification showed central, membrane-bound fenestrae within the isolated caveolae. Central dense, rounded knobs as described originally in vivo (Palade, G. E. and Bruns, R. R., J. Cell Biol. 37:633 (1968)) could be found within some of these fenestrae (unpublished observations).

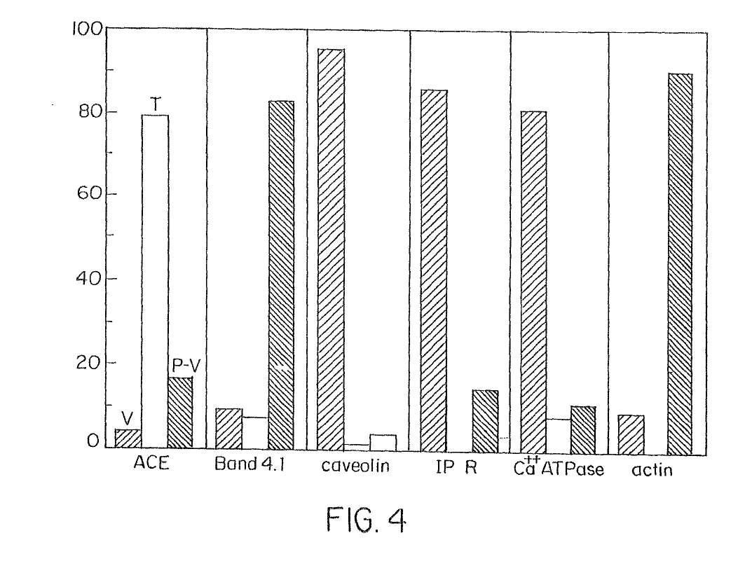

Biochemical analysis revealed a distinct protein profile for the isolated vesicles, with not only very evident enrichment of various proteins relative to the starting membrane pellet but also exclusion of other proteins. Immunoblotting showed a significant enrichment in V for caveolin, plasmalemmal Ca²⁺-ATPase, and IP₃ receptor, with up to a 13-fold enrichment for caveolin and IP₃ receptor relative to original membrane (see Table 1, below). Furthermore, little signal for these proteins remained behind in the silica-coated membrane (P-V) after the caveolae were removed. Immunoblots that were quantified revealed that these three integral membrane proteins were resistant to Triton solubilization and concentrated within the purified caveolae (FIG. 4). Eighty to 95% of the signal for the Ca²⁺ pump (Ca²⁺ ATPase), IP₃ receptor (IP₃R), and caveolin was within the caveolar fraction. By contrast, band 4.1 and ACE were excluded. These purified caveolae represented a microdomain of the plasma membrane with at least three resident proteins that were not just freely distributed over the whole cell surface but preferentially localized to this organelle.

The extent of purification of caveolae is indicated by the relative enrichments for caveolin (see the Table 1, below). As far as yields, it was previously shown that >90% of the microvasculature in situ was coated with silica and at least 80% of the silica-coated membrane was pelleted from the rat lung homogenates (Jacobson, B. S., et al., Eur. J. Cell. Biol. 58:296 (1992)). The recovery of caveolin in the membrane pellet (P) was 10% of the total detected in the starting homogenate, which was consistent with the concept that only the plasmalemmal subset of caveolin-containing vesicles from the luminal side of the endothelium was isolated. The yield of plasmalemmal caveolae derived directly from the original silica-coated membranes (P) ranged from 53% to 60% over four separate experiments as indicated by ELISA and immunoblotting for caveolin. Overall, about 5 μg of total protein were isolated, which represented about 5-6% of the caveolae in the starting lung homogenates.

TABLE 1
Relative Distribution of Various Proteins in Rat Lung Fractions

| | Pellet/ | Vesicle/ |
|---|---|---|
| Antigen | Homogenate | Pellet |
| ACE | 15 | 0.08 |
| Caveolin | 30 | 13 |
| Fibroblast surface antigen | 0 | — |
| Ca²⁺-ATPase | 8 | 3 |
| IP₃ Receptor | 3 | 12 |
| Band 4.1 | 15 | 0.09 |
| Cytochrome oxidase | 0 | — |
| Ribophorin | 0 | — |

Data represent a composite of new experiments/antigens and previously reported results (Jacobson, B.S., et al., Eur. J. Cell. Biol. 58:296 (1992)). Quantified band intensities of immunoblots were normalized per unit of protein before computation of ratios as an average of at least two determinations. The value 0 indicated antigen not detected in P but found in H; — indicates not done.

### Example 3

### Isolation of Microdomains of GPI-Anchored Proteins (G)

The silica coating of the outer membrane surface altered the way in which the GPI-anchored proteins interacted with various detergents and thus prevented the separation of noncaveolar, detergent-resistant microdomains from the cell membranes. Cationic silica particles interact with the anionic cell surface to stabilize it against vesiculation or lateral rearrangement by immobilizing membrane molecules (Chaney, C. K. and Jacobson, B. S., J. Biol. Chem. 258:10062 (1983); Patton, W. F., et al., Electrophoresis 11:79 (1990)). Because the silica particles uniformly coated the cell surface but were rarely associated with or present inside the caveolae because of their size, it is likely (Schnitzer, J. E., et al., Proc. Natl. Acad. Sci. USA 92:1759 (1995); Jacobson, B. S., et al., Eur. J. Cell Biol. 58:296 (1992)) that the plasma membrane was stabilized by being firmly attached on one side to most, if not all, nonvesiculated regions. This adherent pellicle allowed the caveolae on the opposite side of the membrane to be sheared away by homogenization, with little contamination from other membranes, including other detergent-resistant domains. Conversely, without silica coating, both caveolar and non-caveolar detergent-resistant membranes were co-isolated.

Because the silica coating prevented the release of the detergent-insoluble membranes rich in GPI-anchored proteins, it was possible to isolate these domains separately from the caveolae. A method by which this was carried out is represented schematically in FIG. 2. Silica-coated membranes stripped of caveolae (P-V) were incubated with 2 M KH₂PO₄, followed by homogenization in Triton X-100 at 4° C. This procedure allowed the isolation by sucrose density gradient centrifugation of a membrane fraction (G) that contained vesicles of >150 nm in diameter with no caveolae by morphological and biochemical criteria (data not shown).

### Example 4

### Isolation of a Membrane Fraction Comprising Caveolae and Microdomains of GPI-Anchored Proteins (TI)

Methods similar to those described above were used to isolate a membrane fraction which contained caveolae, G domains, and caveolae associated with G domains, as represented schematically in FIG. 3. The salt concentration was increased during the isolation of the silica-coated membrane pellicles, which sufficiently reduced electrostatic interactions between the cationic silica particles and the polyanionic cell surface to detach the plasma membrane from the silica-coated membrane pellicle in (P). Under these conditions, intact membranes were separated, and, with the addition of Triton X-100, low density, detergent-resistant membranes (TI) were isolated by sucrose density gradient centrifugation, as described above.

### Example 5

### Protein Analysis of Various Membrane Subfractions

Differential detergent extraction was performed on silica-coated endothelial cell membranes (P). Equal portions of resuspended P were incubated with rotation for 1 hour at 4° C. with various detergents (β-OG, β-octylglucoside; sodium deoxycholate; Triton X-100) before centrifugation at 13,000 g for 2 hours. The soluble proteins (S) and the sedimented, insoluble proteins (I) were fractionated by SDS-polyacrylamide gel electrophoresis (10 μg/lane), transferred to nitrocellulose or Immobilon (Millipore) filters, and subjected to immunoblot analysis with equivalent amounts of specific antibodies for caveolin, 5′-NT, Band 4.1, GM, and uPAR and the appropriate 125-I-labeled secondary antibodies as described (Schnitzer, J. E. and Oh, P., J. Biol. Chem. 269:6072 (1994); Milci, A. J., et al., J. Cell Biol. 105:2603 (1987)). Other proteins tested included angiotensin-converting enzyme, which was solubilized by all of these detergents, and carbonic anhydrase, which was solubilized similarly to 5′-NT (unpublished data). Proteins from rat lung homogenate (H), the Triton X-100-insoluble membranes isolated by sucrose density gradient centrifugation (TI), and the sedimented pellet (R) were also subjected to immunoblot analysis as above, with the exception that the secondary antibodies were conjugated to horseradish peroxidase (HRP) and binding was detected with ECL chemiluminescent substrate (Amersham).

Detergent extraction studies performed on P revealed differences in the ability of various detergents to solubilize caveolin and 5′-nucleotidase (5′-NT). Caveolin was partially solubilized by P-octyl glucoside, CHAPS, deoxycholate, NP-40, and SDS (but not Triton X-100), whereas 5′-NT was rendered soluble only by SDS and deoxycholate (data not shown). Isolations with rat lung tissue, performed as in Lisanti, M. P., et al. (J. Cell. Biol. 126:111 (1994)), demonstrated that caveolin and GPI-anchored proteins (in this instance, 5′-NT) were both present in the isolated Triton X-100-insoluble membranes (TI) (data not shown), consistent with previous studies (Sargiacomo, M., et al., J. Cell. Biol. 122:769 (1993); Lisanti, M. P., et al., J. Cell. Biol. 123:595 (1993); Change, W.-J., et al., J. Cell Biol. 126:127 (1994); and Lisanti, M. P., et al., J. Cell Biol. 126:111 (1994)). The differential detergent extraction studies performed on TI demonstrated that GPI-anchored proteins were solubilized effectively by P-octyl glucoside, CHAPS, deoxycholate, and SDS, as expected (Brown, D. A. and Rose, J. K., Cell 68:533 (1992); Letarte-Murhead, M., et al., Biochem. J. 143:51 (1974); Hoessli, D. and Runger-Brandle, E., Exp. Cell. Res. 166:239 (1985); Hooper, N. M. and Turner, A. J., Biochem. J. 250:865 (1988); Sargiacomo, M., et al., J. Cell. Biol. 122:789 (1993); Lisanti, M. P., et al., J. Cell. Biol. 123:595 (1993)). This pattern was different from the pattern of solubility for 5′-NT in P but similar to that for caveolin in P.

The lack of GPI-anchored proteins in the purified caveolae enriched in caveolin and ganglioside GM₁ was also demonstrated. The lipid-anchored, cholera toxin-binding ganglioside GM₁ has been localized with gold labeling inside the caveolae (Parton, R. G., J. Histochem. Cytochem. 42:155 (1994); Montessano, R., et al., Nature 296:651 (1982)), and was thus used as a caveolar marker. Whole-lung homogenate (H), silica-coated luminal endothelial membranes (P), purified caveolae (V), and the resedimented silica-coated membranes after stripping of the caveolae (P-V) were subjected to immunoblot analysis as above. GM₁ was detected not only by immunoblotting but also by direct blotting with HRP-conjugated cholera toxin. Purified caveolae enriched in caveolin and ganglioside GM₁ lack GPI-anchored proteins: V contained >90% of GM₁. The remaining membrane devoid of caveolae lacked detectable GM₁ although it was rich in GPI-anchored proteins. Furthermore, like caveolin, 5′-NT and urokinase-plasminogen activator receptor (uPAR) were enriched in P relative to the starting rat lung homogenate (H). However, unlike caveolin, these proteins were not enriched in V; they remained almost totally associated with the resedimented silica-coated membranes stripped of the caveolin (P-V) which contain few, if any, remaining caveolae. More than 95% of the signal for caveolin was detected in V, with <4% remaining in P-V. Conversely, >95% of 5′-NT and uPAR remained in P-V, with <3% present in V. Thus, these GPI-anchored proteins were neither coupled to caveolin nor concentrated in the isolated, caveolin-enriched caveolae. Thus, as with caveolae present on the endothelial cell surface in vivo (Kurzchalia, T. V., et al., J. Cell Biol. 118:1003 (1992); Dupree, P.1, et al., EMBO J. 12:1597 (1993); Rothberg, K. G., et al., Cell 68:673 (1992); Fujimoto, T., et al., J. Cell. Biol. 119:1507(1992); Fujimoto, T., J. Cell. Biol. 120:1147 (1993)), the purified caveolae (V) were enriched in caveolin, plasmalemmal Ca²⁺-dependent adenosine triphosphatase, and the inositol 1,4,5-triphosphate receptor. In contrast, other markers present amply in P, including angiotensin-converting enzyme, band 4.1, and β-actin, were almost totally excluded from V.

The GPI-anchored protein which was isolated separately from the silica-coated membranes was also subjected to immunoblot analysis, performed as described above, with P-V, from the silica and RP (the resedimented pellet of silica-containing material). The silica-coated membrane pellet already stripped of caveolae (P-V) was resuspended in 20 mM 2-IN-morpholino) ethenesulfonic and with 125 mM NaCl and an equal volume of 4 M K₂HPO₄ and 0.2% polyacylate (pH 9.5). The solution was sonicated (10 10-s bursts) with cooling, mixed on a rotator for 8 hours at room temperature (20° C.), and sonicated again (five 10-s bursts). Triton X-100 was added to 1%, and the preparation was then mixed for 10 min at 4° C. and homogenized with a Type AA Teflon tissue grinder (Thomas Scientific, Swedesboro, N.J.). Any intact floating detergent-resistant membranes were separated and isolated from this homogenate by sucrose density gradient centrifugation as above. The caveolin in P-V represents the small residual signal after stripping of the caveolae (compare V and P-V). GM₁ could not be detected in P-V, nor, as expected, in G or RP (data not shown). G is rich in GPI-linked proteins (5′-NT, uPAR and CA) but lacks caveolin and GM₁. Control experiments performed identically but without high salt did not yield any detectable membranes in the sucrose gradient (data not shown). G lacked caveolin but was enriched in several GPI-anchored proteins: 5′-NT, uPAR, and carbonic anhydrase (CA). These results demonstrated that distinct detergent-resistant plasma membranes rich in GPI-anchored proteins but lacking caveolin were isolated separately from the caveolae. Similar detergent-resistant membranes, consisting of large vesicles rich in GPI-anchored proteins but devoid of caveolin, have also been isolated from lymphocytes and neuroblastoma cells, both of which lack caveolae and do not express caveolin (Fra, A. M., et al., J. Biol. Chem. 269:30745 (1994); Gorodinsky, A. and Harris, D. A., J. Cell Biol. 129:619 (1995)).

Immunoblot analysis of caveolae isolated without Triton X-100 was also conducted. Caveolae were purified without any exposure to detergent. As noted above, caveolae can be isolated without exposure to Triton X-100, but less efficiently. The usual protocol was followed for caveolae isolation, with the exception that Triton X-100 was omitted and, for shearing purposes, the number of homogenization strokes was increased to 48 to 60 from 12). These caveolae (V′) and the membrane stripped of them (P′-V′) were subjected to immunoblot analysis as described above. The results were consistent with those obtained from caveolae purified without detergent: caveolin and GM₁ were enriched in V′ whereas GPI-anchored proteins were almost completely excluded from detergent-free purified caveolae (V′).

Other studies that have examined membrane diffusion by fluorescence recovery after photobleaching have detected a larger fraction of GPI-anchored proteins (20 to 60%) present in an immobile fraction (Hannan, L. A., et al., J. Cell. Biol. 120:353 (1993); Brown, D. A. and Rose, J. K., ell 68:533-544 (1992); Zhang, F., et al., J. Cell. Biol. 115:75 (1991); Zhang, F., et al., Proc. Natl. Acad. Sci. USA, 89:5231 (1992)). By examination of detergent solubility, similar percentages have been detected for the GPI-linked proteins in the detergent-resistant microdomains, suggesting equivalence of this fraction with the immobile fraction detected in the diffusion studies. Specialized glycolipid domains are resistant to detergent extraction and are necessary for maintaining detergent-resistant clusters of GPI-linked proteins (Schroeder, R., et al., Proc. Natl. Acad. Sci. USA. 91:12130 (1994); Brown, D. A. and Rose, J. K., Cell 68:533 (1992); Letarte-Murhead, M., et al., Biochem. J. 143:51 (1974); Hoessli, D. and Runger-Brandle, E., Exp. Cell. Res. 166:239 (1985); Hooper, N. M. and Turner, A. J., Biochem. J. 250:865 (1988); Sargiacomo, M., et al., J. Cell. Biol. 122:789 (1993); Lisanti, M. P., et al., J. Cell. Biol. 123:595 (1993)). Removal of cholesterol from plasma membranes can dissociate or prevent the formation of such clusters and assure a random, free distribution of GPI-anchored proteins (Rothberg, K. G., et al., J. Cell Biol. 111:2931 (1990)). As expected, cholesterol removal reduces the resistance of GPI-linked proteins to detergent solubilization (Sargiacomo, M., et al., J. Cell. Biol. 122:789 (1993); Lisanti, M. P., et al., J. Cell. Biol. 123:595 (1993)), consistent with the notion that the freely diffusing GPI-anchored proteins are indeed more readily solubilized by detergent than the less mobile GPI-anchored proteins in the glycolipid domains. Moreover, in the absence of glycolipids, GPI-anchored proteins are readily solubilized from membranes by cold Triton X-100; solubility decreases with the addition of appropriate glycolipids (Schroeder, R., et al., Proc. Natl. Acad. Sci. USA. 91:12130 (1994)). Thus, GPI-anchored proteins randomly distributed at the cell surface should be susceptible to detergent extraction; indeed, the percentages shown herein agree with those from the diffusion studies. In homogenates of non-silica-coated rat lung, approximately 60% of CA and 75% of 5′-NT are solubilized by Triton X-100 at 4° C. Moreover, mass balances performed on the silica-coated membranes showed that approximately 20% of 5′-NT and 40% of CA could be isolated in the intact, detergent-resistant membrane fraction TI.

Thus, it appears that a substantial but variable fraction of GPI-anchored proteins exists on the cell surface dynamically partitioned into detergent-resistant glycolipid microdomains that are not likely to be simply a consequence of detergent extraction, and that the size of this fraction may depend on cell type, culture, and ligand or antibody exposure.

Lipid anchors such as GPI may control the ability of proteins to partition selectively, but reversibly, within the specialized microdomains and, therefore, may subserve a targeting function. The GPI anchor directly affects association with detergent-resistant membranes (Rodgers, W., et al., Mol. Cell. Biol. 14:5364 (1994)), membrane diffusion (Hannan, L. A., et al, J. Cell. Biol. 120:353 (1993); Zhang, F., et al., J. Cell. Biol. 115:75 (1991); Zhang, F., et al., Proc. Natl. Acad. Sci. USA, 89:5231 (1992)), polarized delivery to cell surfaces (Brown, D., et al., Science 245:1499 (1989); Simons, K. and van Meer, G., Biochemistry 27:6197 (1988); Garcia, M., et al., J. Cell Sci. 104:1281 (1993)), cell activation (Su, B., et a., J. Cell Biol. 112:377 (1991)), and the rate and pathway of internalization (Keller, E.-A., et al., EMBO J., 3:863 (1992)). Other lipid-associated proteins, including NRTKs and guanosine triphosphate (GTP)-binding proteins such as Rab5, are found in the detergent-resistant complexes (Sargiacomo, M., et al., J. Cell. Biol. 122:789 (1993); Lisanti, M. P., et al., J. Cell. Biol. 123:595 (1993); Chang, W.-J., et al., J. Cell. Biol. 126:127 (1994); Lisanti, M. P., et al., J. Cell. Biol. 126:111 (1994); Rodgers, W., et al., Mol. Cell. Biol. 14:5364 (1994); Arreaza, G., et al., J. Biol. Chem. 269:19123 (1994); Shenoy-Scaria, A. M., et al., J. Cell Biol. 126:353 (1994)). The current analysis reveals that various NRTKs (Yes and Lyn, unpublished data) heterotrimeric GTP-binding proteins (α and βγ subunits) (Schnitzer, J. E., et al., J. Biol. Chem. 270:14399 (1995)), and as yet unidentified small GTP-binding proteins, but not Rab5 (Schnitzer, J. E., et al., J. Biol. Chem. 270:14399 (1995)), are indeed present in purified caveolae.

### Example 6

### Electron Microscopy of V and TI Preparations

Preparations V and TI were examined under electron microscopy to determine whether caveolae were equivalent with low density, Triton insoluble membranes. The electron microscopy was performed on membrane isolates as described in Schnitzer, J. E. et al., Proc. Natl. Acad. Sci., USA, 92:1759-1763 (1995), the teachings of which are incorporated herein by reference.

Electron microscopy of the vesicles (V) purified from the silica-coated rat lung endothelial membranes showed that the V isolate shows a homogenous population of small vesicles (≦100 nm) with typical caveolar morphology. Despite the isolation procedure, many caveolae retained their characteristic flask shape.

The detergent-resistant membranes (TI) isolated without silica coating consisted of many larger vesicles (>150 and <700 nm in diameter) interspersed with smaller caveolar vesicles (<100 nm) and some nonvesiculated, linear membrane sheets. A typical caveola was often apparent attached to the inside of a larger vesicle. In many favorable cross-sections, a characteristic flask-shaped caveola attached to a larger, spherical vesicle was apparent, suggesting that these two detergent-resistant membrane domains were associated with each other as a unit before fractionation in the membrane.

### Example 7

### Colloidal Gold Immunolabeling

Detergent-resistant membrane isolates (TI) were embedded in agarose for gold labeling of CA or GM₁. The lipid-anchored molecule, the cholera toxin-binding ganglioside GM₁, has been localized with gold labeling inside the caveolae (Parton, R. G., J. Histochem. Cytochem. 42:155 (1994); Montesano, R., et al., Nature 296:651 (1982)), and was therefore used as a marker for caveolae. Overall, a size criterion was obvious in distinguishing the caveolar vesicles from the noncaveolar vesicles. Therefore, the vesicles clearly observed in the electron micrographs were divided into two groups: those with diameters of <80 nm and those with diameters of >150 nm. This size criterion cannot be considered absolute in separating caveolae from noncaveolar vesicles, because, for instance, a few caveolae could remain attached to each other and form a larger vesicle. Nevertheless, the results of immunolabeling supported the use of GM₁ as a caveolar marker, and substantiated the size criterion.

At low magnification, immunogold labeling of TI localized CA primarily on the surface of the larger vesicles and linear membranes but not on the smaller caveolae. All gold is attached to membranes with little, if any, background labeling. At higher magnification, images revealed unlabeled caveolae apparently attached to large vesicles labeled with gold or associated with labeled membrane strands attached to the neck of the caveolae. Control experiments with nonimmune serum showed little labeling of membranes; only an occasional gold particle was detected per field examined and appeared equivalent to background labeling of agarose alone. In contrast, higher magnification micrographs revealed that immunogold labeling for GM₁ was frequently detected inside the caveolae, with little labeling of the caveolae-associated larger vesicles or remnant membranes. This result was consistent with the biochemical data and with gold localization of GM₁ performed on cells (Parton, R. G., J. Histochem. Cytochem. 42:155 (1994); Montesano, R., et al., Nature 296:651 (1982)). Control experiments performed with conjugates plus a 10-fold molar excess of monomeric cholera toxin showed almost complete absence of gold.

Although several previous studies that have examined the immunolocalization of GPI-anchored proteins in cultured cells concluded that these proteins reside in caveolae (Rothberg, K. G., et al., J. Cell. Biol. 110:637 (1990); Ying, Y., et al., Cold Spring Harbor Symp. Quant. Biol. 57:593 (1992); Ryan, U. S., et al., J. Appl. Physiol. 53:914 (1982); Stahl, A. and Mueller, B. M., J. Cell Biol. 129:335 (1995)) reexamination of the published electron micrographs reveals little gold labeling directly inside the caveolae. Almost all of this labeling is actually adjacent to the caveolae on the flat plasma membrane directly attached to, but not a part of, the neck of the caveolae. The small amount of labeling apparent inside the caveolae and the extent of clustering observed may be induced artifactually by antibody cross-linking (Mayor, S., et al., Science 264:1948 (1994)). The data provided herein confirm that GPI-anchored proteins are not within the caveolae, but are attached to membrane that is adjacent to the caveolae.

This is not meant to imply that GPI-anchored proteins can never enter the caveolae. Antibody-cross-linked alkaline phosphatase clusters and slowly enters caveolae for endocytosis to endosomes and lysosomes (Parton, R. G., et al., J. Cell Biol. 127:1199 (1994)), consistent with studies of internalization of modified albumins by caveolae, with the exception that the process of binding, clustering, internalization, and degradation was much quicker to the albumin (Oh, P., et al., J. Cell Biol. 127:1217 (1994); Schnitzer, J. E., et al., J. Biol. Chem. 264:24544 (1992); Schnitzer, J. E. and Bravo, J., J. Biol. Chem. 268:7562 (1993)). It appears that cell surface processing, at least for GPI-linked proteins, probably comprises three distinct sequential steps: (i) induced movement of GPI-anchored proteins (probably by a ligand) into microdomains near the caveolae, thereby increasing the local concentration of GPI-linked proteins by direct sequestration of previously free molecules or possibly by assembly of several small clusters; (ii) eventual movement into the caveolae; and (iii) fission or budding of the caveolae from the membrane for photocytosis or endocytosis.

### Example 8

### Monoclonal Antibodies Generated to Plasma Membranes and Caveolae of Endothelium in situ

The luminal endothelial cell surface is the critical interface interacting directly with the circulating blood to maintain cardiovascular homeostasis by helping to mediate many functions including vascular tone, capillary permeability, inflammation and coagulation. This surface is directly accessible to drugs injected intravenously and may contain useful organ-specific endothelial targets for selective drug and gene delivery. Furthermore, caveolae found abundantly on the surface of many endothelia provide a vesicular pathway for trafficking blood molecules and possibly vascular targeting drugs into and across the endothelium.

Luminal endothelial cell plasma membrane was purified along with its caveolae directly from rat lung tissue by in situ coating procedures (Science 269:1435-1439 (1995)) in order to generate specific monoclonal antibodies (mAbs). Monoclonal antibodies were generated by standard techniques, using 100 μg of P as immunogen. Over 100 hybridomas were raised that recognize by ELISA the silica-coated luminal endothelial cell plasma membranes adsorbed onto 96-well trays. Twenty stable clones were established and their mAbs analyzed by Western blotting and tissue immunocytochemistry.

Two of the antibodies were eliminated from further study because they were not successful during Western blotting, probably as a consequence of protein denaturation since they did recognize antigen in the silica-coated plasma membranes by ELISA and in tissue sections by immunomicroscopy.

Three of the antibodies (167, 278, 461) did not recognize any molecules in caveolae, as their signal was found only in (P) and (P-V). Seven mAbs (833, 472, 154, 228, 302, 309 and another mAb) recognized antigens found primarily in purified caveolae (V), based on their enrichment in (V) but near complete absence from (P-V). Two of these antibodies (833 and 472) reacted with the surface of microvascular endothelium in lung tissue, as assessed by both immunoblotting and tissue immunostaining. Antibodies 833 and 472 appeared to be monospecific for proteins in P of about 85 and 90 kDa, respectively. Densitometry revealed that both antigens were very enriched in P over the starting lung homogenate (H) (78-fold for 833, and 23-fold for 472) and also in the purified caveolae (V) over the silica-coated membrane stripped of caveolae (P-V) (60-fold for 833 and 7-fold for 472). In addition, significant co-localization was found on the cell surface of the signal of 833 and 472 with that of caveolin recognized by commercially available antibodies.

All of the other antibodies recognized specific proteins enriched in the silica-coated endothelial cell plasma membranes (P) over the starting lung homogenates (H). In many cases, it was difficult to detect the antigen in the starting lung homogenates, but the antigen was readily apparent in P, reflecting significant enrichment provided by the purification.

Western analysis was performed on whole tissue lysates, as described previously, using 20 μg of proteins solubilized from the indicated tissues (Schnitzer, J. E. et al., Science 269:1435-1439 (1995); Schnitzer, J. E. et al., J. Biol. Chem. 270:14399-14404 (1995); Schnitzer, J. E. and Oh, P., J. Biol. Chem. 269:6071-6082 (1994)). Because it is likely that some of the antigens, especially those expressed exclusively in endothelia, might not be detected by Western analysis of whole tissue lysates, various rat tissues were also screened by immunohistochemistry and microscopy. To perform immunostaining, rat organs were flushed free of blood, and then fixed by perfusion of cold 4% paraformaldehyde in PBS. The lung parenchyma was expanded by filling the bronchus with OCT compound (Miles; Elkhart, Ind.). Small tissue samples were fixed in paraformaldehyde for 2-3 hours, infiltrated with cold 30% sucrose overnight and then frozen in OCT at −70° C. Frozen 5 μm sections were cut and placed on poly-l-lysine coated glass slides. At room temperature, the sections were dried for 30 minutes, washed for 10 minutes in PBS, treated with 0.6% hydrogen peroxide in methanol for 10 minutes, washed again, blocked for 30 minutes with 5% sheep serum, and then incubated for one hour with 1 μg/ml of purified Igg. After washing, the tissue sections were incubated for 30 minutes with 10 μg/ml of biotin-labeled sheep anti-mouse IgG (The Binding Site; Biurmingham, U<) in blocker, washed, incubated for 20 minutes with 1 μg/ml of streptavidin conjugated to horseradish peroxidase (Biogenex; San Roman, Calif.) in blocker. After washing, the tissue was incubated for 3-5 minutes with enzyme substrate 3-amino-9-ethylcarbazole (Zymed, South San Francisco, Calif.) before rinsing away substrate with distilled water. The tissue was counter-stained with Hematoxylin, rinsed, dried, and covered with cover slip using mounting gel (Biomeda; Foster, Calif.).

Based on the initial tissues tested (lung, heart, brain, liver, kidney, adrenal, testes, intestine, skeletal muscle, and spleen), both Western blotting of whole tissue lysates and immunohistochemical staining of formaldehyde-fixed tissue sections indicated good lung specificity for both 833 and 472. They reacted with the endothelium only in lung tissue and did not stain larger blood vessels, suggesting microvascular specificity. The lung epitherlial cells, especially obvious in the bronchi, were also nonreactive. In contrast, many of the other monoclonal antibodies stained endothelia in many different tissues and in both large and small blood vessels. Screening of rat organs indicated that most of these mAbs recognized all endothelia, whereas a few were specific for continuous endothelia. Because mAb 167 was reactive with screened serum by ELISA, it was removed from further immediate investigation.

The more organ- and caveolae-specific antibodies (833, 472, 154, 228, 302 and 309) are being examined further, as is mAb 881 which recognizes all tissue endothelia and acts as a positive control. The two lung-specific antibodies, mAb 833 and 472, were used to assess immunotargeting in vivo. mAb 833 and 472 IgG were purified, and radio-iodinated with lodogen, and desaled to remove free ¹²⁵I. 500 μl containing 10 μg of labeled IgG diluted in 10 mg/ml of rat serum albumin (Sigma; St. Louis, Mo.) was injected into the tail veins of male Sprague-Dawley rats (150-200 g) to assess immunotargeting in vivo. A negative control antibody recognizing vinculin, an antigen not exposed at the endothelial cell surface, was also used. After 30 minutes the rats were anesthetized terminally using 300 U Ketamine and 10 mg Xylazine before thoracotomy and blood removal (1 ml) by cardiac puncture. The indicated tissues were removed and weighed before counting gamma radioactivity. The amount of antibody per gram of tissue was calculated using the specific activity of each antibody. In the first set of rats studied, ⁵¹Cr-labeled red blood cells were also injected in order to determine actual tissue uptake by determining and then subtracting tissue blood volumes (Bernareggi, A. and Rowland, M., J. Pharmacokin. Biopharm. 19:21-50 (1991)). Because of the low blood concentrations, this correction was negligible relative to the mAb 833 and 472 detected in the tissues. Therefore, the practice was discontinued.

In just 30 minutes, very different biodistributions for each antibody appeared. Each different antibody was injected into three different rats, with nearly identical results. The nontargeting negative control antibody had low reactivity and remained primarily intravascular, as indicated by very high counts in the blood. As expected, the liver had the most significant uptake of this antibody. Conversely, both 472 and 833 had very low blood counts (>10-fold less than the control for 833) and very significant tissue uptake in the lung (>50-fold for 833 over the control). Both showed similar low levels of uptake in the liver, comparable to the control antibody, as would be expected for all antibodies not recognizing an alternative target in the liver. Most importantly, mAb 833 appeared specifically to accumulate most rapidly and significantly in the lung with very little detection in other organs. Mass balance analysis showed that a mean of 75±6.4% (833; ranging from 67 to 87%) and 16±1.1% (472) of the injected dose (10 μg each), is targeted to the lung tissue in just 30 minutes. Both results are in sharp contrast to the 1.4% of the nontargeted antibody found in the lung tissue. Both of these targeting antibodies significantly exceeded part reports using various targeting monoclonal antibodies requiring up to one week to achieve a maximal tissue uptake of 0.2-4% of the injected dose (Tomlinson, E., Advanced Drug Delivery Reviews 1:87-198 (1987); Ranney, D. F., Biochem. Pharmacol. 35:1063-1069 (1986); Holton, O. D. et al., J. Immunol. 139:3041-3049 (1987); and Pimm, M. V. and Baldwin, R. W., Eur. J. Clin. Oncol. 20:515-524 (1984)). Even 24 hours after injection, 46% of the injected dose of mAb 833 still remained in the lung tissue, consistent with the expected transport into or across the endothelium by the caveolae (Schnitzer, J. E., Trends in Cardiovasc. Med. 3:124-130 (1993); Schnitzer, J. E. and Oh, P., J. Biol. Chem. 269:6072-6082 (1994); Schnitzer, J. E. et al., J. Cell. Biol. 1271217-1232 (1994)).

To put the results into perspective, the accepted pharmaceutical criterion for assessing drug localization and targeting is that the therapeutic index must increase by half a log unit or about 3-fold. thus, a true targeting method should cause the usual levels in nontarget organs to rise less than one-third of the lung increment (Ranney, D. F., Biochem. Pharmacol. 35:1063-1069 (1986)). For mAb 833, the uptake in the non-lung organs changed minimally or even decreased from the control IgG while the lung uptake increased by 50-fold. More stringent evaluation by calculation of the tissue targeting index (TTI) (antibody in tissue/g of tissue/antibody in blood/g or blood) and tissue selectivity index (TSI) (TTI for the targeting IgG in tissue/TTI for nontargeting control IgG in the same tissue) revealed excellent targeting by 833 with exquisite selectivity for the lung with a mean TTI of 56 and a mean TSI of 150. mAb 472 gave an excellent TTI for lung (34), but also targeted several other tissues (spleen, TTI=8.2, kidney, TTI=4.1, and adrenals, TTI=4.6). In contrast, the control IgG lacked significant targeting with TTI<1 for all organs examined and a maximum value for liver at 0.36. Lastly, the rapid selective tissue targeting observed with 833 did not require intravenous injection, and could easily be detected when injected arterially so that passage through the systemic circulation occurred first before reaching the lungs. Comparisons of injections of mAb 833 directly into the right or left ventricular chambers in thoracotomized rats demonstrated that even after only 15 minutes in the circulation, the TTI for both injections already exceeded 10 (11 for left ventricular chamber, and 16 for right ventricular chamber) while being >1 for all other tissues examined including the liver. This level of tissue specifity, targeting and rapidity of uptake is unprecedented.

In light of the in vivo findings, further tissue immunostaining studies were conducted to include the non-lung tissues exhibiting uptake of 472. A weak signal was detected in formaldehyde-fixed spleen but not other tissues. When specimens were fixed more mildly with acetone, it was evident that mAb 472 stained blood microvessels in lung, kidney, adrenal gland and spleen, but not heart, liver, intestine, brain, muscle and testes. This tissue distribution supports the above izn vivo findings. Again, mAb 833 stained microvascular endothelium only in the lung.

These organ-specific antibodies demonstrate that accessible, organ-specific targets exist on the endothelial cell surface in vivo, and provide a means for localization or targeting of agents to specific organs or tissues, including targets in normal and diseased tissues.

### Example 9

### Targeting Endothelium and Dynamic Caveolae for Tissue-Specific Transcytosis

Methods

Antibody Production.

Monoclonal antibodies were generated by standard somatic cell hybridization using 100 μg of silica-coated luminal endothelial cell plasma membranes (P) as an immunogen and were screened by ELISA with P adsorbed onto 96-well trays.

In vivo Biodistribution Studies.

IgG was purified by Protein G chromatography (Pierce, Rockford, Ill.) and radiolabeled with ¹²⁵I using lodogen (25). The rat tail vein was injected with 10 μg of ¹²⁵I-IgG in 500 μl rat serum albumin (10 mg/ml). After 30 min, the rats were anesthetized for thoracotomy, blood sampling by cardiac puncture, and organ removal. Tissues were weighed before counting gamma radioactivity to determine antibody/g of tissue. In initial studies, ⁵¹Cr-labeled red blood cells were injected to determine actual tissue uptake by subtracting tissue blood volumes. For TX3.833, this correction was negligible and the practice discontinued.

Antibody-Au Conjugates.

A monodispersed solution of colloidal gold (avg. diam. 6 nm) (EM Sciences, Fort Washington, Pa.) was adjusted to pH 9.2 with K₂CO₃ before purified IgG and stirring rapidly for 30 min. PEG (Mr 20,000) was added to a concentration of 0.5 mg/ml for the last 5 min. After centrifugation at 105,000× g for 1 h at 4° C., the loose pellets were collected and resuspended in 5 mM phosphate before dialysis against 50 mM Tris during which NaCl was added slowly to a concentration of 150 mM. The conjugates were used within 48 h of preparation.

Tracking Antibody-Au Complexes Perfused in situ and in vivo.

For the in situ experiments, the cranial lobe of the rat lung was perfused at 10 mmHg with PBS+ (PBS containing 3% BSA and 14 mM glucose) at 37° C. followed by 6 ml of TX3.833-Au or control mouse IgG₁-Au in PBS+ (OD₅₄₀=18). Pulmonary artery perfusion was limited to the cranial lobe by ligature exclusion of all other lobes of the lung. After 2, 5, 10, or 15 min, the lobe was flushed with 6 ml PBS+ at 37° C. before perfusion fixation with 2% paraformaldehyde and 2.5% glutaraldehyde in 0.1M Na cacodylate (KII). The removed lobe was processed for Epon embedding and electron microscopy (EM) as described in (Oh, P. et al., J. Cell Biol. 141:101-114 (1998)). To track TX3.833-Au in vivo, a TX3.833-Au (15 nm) conjugate (500 μg Ab) was injected directly into rat tail veins. After 15 min the rat was given intramuscular anesthesia and, as described above, the lungs were flushed with PBS+ followed by KII and processed for EM (Oh, P. et al., supra).

Morphometry.

Randomly selected fields were examined and recorded at a final magnification of 49,500×. As described (Milici, A. J. et al., J. Cell Biol. 105:2603-2612 (1987); Oh, P. et al., J. Cell Biol. 141:101-114 (1998)), an image analyzer (Analytical Imaging Concepts, Roswell, Ga.) was used to determine the number of gold particles in caveolae/unit surface of linear plasma membrane (4 μm) and in the interstitium/unit surface area (μm²) of each compartment. The number of gold particles found in >150 well-defined, apparently single caveolae (no other connecting caveolae or part of one visible in the section) that were clearly attached to the luminal or abluminal plasma membrane by their necks, were quantified. To minimize variability from sectioning, only caveolae whose diameter exceeded 50 nm were used.

Immuno-Conjugations.

Conjugation of ¹²⁵I to antibody was performed using lodogen (Fraker, P. J. and Speck, J. C., Biochem. Biophys. Res. Comm. 80:849-857 (1978)). Deglycosylated Ricin A chain (dgRA) (Sigma, St. Louis, Mo.) was radio-iodinated (Fraker, P. J. and Speck, J. C., supra), reduced with 5 mM dithiothreitol and filtered through a Sephadex G25 (Pharmacia; Piscataway, N.J.) column in phosphate-EDTA buffer (pH 7.5). N-succinimidyl-3-(2-pyridyldithio)-propionate (SPDP) and sulfosuccinimidyl 4-(N-maleimidomethyl)cyclohexane-1-carboxylate (SMCC) (Pierce, Rockford, Ill.) were used to conjugate dgRA or ¹²⁵I-dgRA to antibody by disulfide linkage or thioether linkage, respectively (Cumber, A. J. et al., Methods in Enzymology 112:207-225 (1985)). The antibody conjugates were Protein G affinity-isolated.

Results

Novel Antibody Specific for Lung Caveolae.

To identify tissue-specific vascular targets and to address whether caveolae can function in selective transport in vivo, mouse monoclonal antibodies were generated to rat lung P and their attached caveolae (Schnitzer, J. E. et al., Science 269:1435-1439 (1995); Oh, P. and Schnitzer, J. E., in Cell Biology: A Laboratory Handbook, ed. Celis, J. (Academic Press, Orlando), Vol. 2, pp. 34-36 (1998)). Screening identified an IgG₁ monoclonal antibody, TX3.833, that appears to be both tissue- and caveolae-specific. Immunoblotting revealed that TX3.833 specifically recognizes a 90 kDa protein expressed in lung but not in other tissues and enriched in lung P relative to the tissue homogenates (H). Further subfractionation of lung P to isolate caveolae (V) showed that this antigen is concentrated in caveolae similar to caveolin-1 but unlike the lipid raft marker, 5′nucleotidase (5′NT). Densitometry performed under equal protein loading conditions revealed >15-fold enrichments for this antigen both in P relative to H and in V relative to P (>225-fold in V relative to H) (N=3). TX3.833 antigen was also detected in caveolin-1-coated caveolae isolated from V using caveolin-1 antibodies (Oh, P. and Schnitzer, J. E., J. Biol. Chem. 274:23144-23154 (1999)) (data not shown).

Antibody mapping of P reveals considerable molecular diversity of endothelia among organs with differential expression of transferrin receptor, thrombomodulin, 5′NT, and caveolin-1. TX3.833 antigen is only detected in P from lung and not other organs. Rat tissue immunostaining confirmed TX3.833 reactivity in lung alveolar microvasculature but not in bronchial epithelium, larger pulmonic vessels, or in any blood vessels of the heart, liver, brain, kidney, intestine, skeletal muscle, testes, of lung spleen, skin and adrenal (data not shown). More importantly, immunogold EM carried out on ultrathin frozen lung tissue sections showed that TX3.833 associated predominantly with the bulb and necks of the caveolae in microvascular endothelium and not clathrin-coated pits or epithelial cells (including their caveolae). Larger blood vessel endothelium and controls using heart tissue or nonspecific mouse IgG₁ were negative (data not shown). Thus, TX3.833 specifically recognizes a 90 kDa antigen that is expressed selectively in caveolae of microvascular endothelium of lung but not other tissues.

Tissue Targeting in vivo.

To assess possible tissue-specific immunotargeting in vivo, purified radio-iodinated TX3.833 or control IgG₁ was injected into rat tail veins. Different biodistributions for each antibody were quite apparent 30 min after injection. TX3.833 showed rapid and substantial lung uptake with very little detected in other organs (values≦to control IgG) and very low blood levels (10-fold<control). Up to 89% with a mean of 75±6.4% of the injected dose of TX3.833 was targeted to the lungs in just 30 min. As in past reports (Holton, O. D. et al., J. Immunol. 139:3041-3049 (1987)), the control IgG remained in the blood with low tissue uptakes; the liver had the most, apparently due to IgG sequestration by F_{c} receptors. The lung tropism of TX3.833 was specific because unlabeled TX3.833, but not control IgG, inhibited lung accumulation by 89% and increased blood levels by >15-fold. Other antibodies generated in the screen recognized endothelial cell surface antigens in several tissues and accumulated in vivo in multiple tissues (data not shown). The uptake of TX3.833 significantly exceeds past reports describing various targeting probes (peptides and monoclonal antibodies) sometimes requiring up to 1 week to achieve a maximal tissue uptake of 0.2-4% of the injected dose (Tomlinson, E., Advanced Drug Delivery Reviews 1:87-198 (1987); Weinstein, J. N. and van Osdol, W., Int. J. Immunopharmacol. 14:457-463 (1992); Holton, O. D. et al., J. Immunol. 139:3041-3049 (1987); Pimm, M. V. and Baldwin, R. W., Eur. J. Clin. Oncol. 20:515-524 (1984); Arap, W. et al., Science 279:377-380 (1998); Hughes, B. J. et al., Cancer Res. 49:6214-6220 (1989); Pasqualini, R. and Ruoslahti, E., Nature 380:364-366 (1996); Chistofidou-Solomidou, M. et al., Am. J. Physiol. Lung Cell Mol. Physiol. 278:L794-805 (2000)). Even 24 hours after injection, 46% of TX3.833 still remained in the lung.

Calculation of the tissue targeting index (TTI=antibody in tissue/g of tissue/antibody in blood/g of blood) and tissue selectivity index (TSI=TTI for targeting IgG/TTI for control IgG) confirmed lung targeting of TX3.833 with a mean TTI of 56 and TSI of 150. The control IgG lacked targeting with TTI<1 for all organs examined (maximum in liver of 0.36). Last, TX3.833 injections into the right vs. left ventricular chambers even after 15 min produced for both injections a TTI>10 for lung and <1 for other tissues. Thus, TX3.833 lung tropism depends not on first pass through the pulmonic circulation but rather on the antigen expression restricted to lung microvascular endothelium.

Transcytosis of Antibody Targeting Lung Caveolae in situ.

To assess possible targeting to and transport by caveolae of TX3.833, TX3.833 conjugated to colloidal gold particles (TX3.833-Au) was perfused through the rat pulmonary artery. EM and morphometric analysis revealed specific and rapid TX3.833-Au targeting to caveolae followed by transendothelial transport of the targeted cargo. Within 2-3 min, TX3.833-Au was found at the endothelial cell surface mostly bound to accessible luminal caveolae, either at their necks (on or near the diaphragm) or penetrating into the bulb of the caveolae. Very little to no TX3.833-Au was detected in caveolae located further inside the cell, attached to the abluminal cell surface, in clathrin-coated pits and vesicles, or in the subendothelial space. After 5 min, penetration of TX3.833-Au into the caveolar system increased with many more caveolae containing gold particles including those connected at the luminal surface, further inside the cell, and at the abluminal surface. Occasionally, gold particles were seen inside (and not yet exiting) abluminal caveolae which opened onto the subendothelial space. By 10 min, many gold particles were detected exiting the endothelium from the abluminal caveolae into the subendothelial space. The amount of gold in the perivascular space was increasing but remained mostly in the proximity of the basal/abluminal endothelial cell plasma membrane (subendothelial space). Gold particles were also observed in racemose caveolar structures. There was little evidence of caveolae-mediated endocytosis to intracellular organelles such as endosomes. Although sometimes TX3.833-Au was seen accumulating at a luminal region near endothelial intercellular junction, little to no gold was found in the interstitium at the junctional exit, consistent with a lack of transport through this pathway and the expected size-exclusion of the gold particles. After 15 min, TX3.833-Au was still being transported and existed in many luminal, abluminal and apparently internalized caveolae. Gold particles continued to accumulate in the interstitium of the tissue.

In several fortuitous cases, the release of gold particles from the neck or introit of caveola was caught. The gold particles were seen as a cluster in the region immediately adjacent to the caveolar opening, apparently exiting as a bolus and just beginning to disperse freely into the underlying space. For example, clusters were found on the plasmalemma proper that sometimes were at or near the caveolar diaphragm. Sometimes 1 or 2 gold particles were still found just inside the neck of an otherwise empty abluminal caveola. No other gold particles were seen in the vicinity. Morphometric analysis showed that a similar number of gold particles entered luminal caveolae as exited abluminal caveolae. At 2.5 minutes, the labeled luminal caveolae clearly attached to the plasma membrane had a mean of 6.4±0.5 particles/caveola with <25% of total luminal caveolae exhibited no labeling or occasionally 1 or 2 gold particles. By 10 min, the mean number of gold particles inside labeled luminal and abluminal caveolae was 6.3±0.3 and 7.1±0.6, respectively, although again, a subpopulation of caveolae (<25%) still had little to no label. Lastly, the mean number of gold particles exiting the neck of abluminal caveola was 6.7±0.8 particles. This quantal uptake, transport, and release of 6-7 gold particles/caveolae is consistent with discrete transcytosis of targeted molecular cargo by caveolae.

By comparing TX3.833-Au with physically identical probes having different binding specificity (i.e. all 150 kDa IgG₁ bound in the same way to the same size gold particles), it was found that the ability of TX3.833 to specifically target the gold to caveolae mediates its transport across the lung microvascular endothelium. Control mIgG-Au, even after 15 min did not accumulate in caveolae nor overcome the endothelial cell barrier. Most examined fields lacked any gold particles and when detected, they were predominantly clustered on flat regions of the plasma membrane as well as near the luminal opening or diaphragm of some caveolae. Few gold particles were found at cell surface, inside the cell and in the perivascular space, consistent with the expecte dexclusion of the probe from the interstitium by the endothelial cell barrier and the inability of the probe to target caveolae. Morphometric analysis showed that both entry to caveolae and interstitial accumulation were significantly more for TX3.833-Au than mIgG-Au. As an additional control, gold-conjugated antibody to 5′NT (5′NT-IgG-Au), a GPI-anchored endothelial cell surface marker concentrated in lipid rafts but not caveolae (Schnitzer, J. E. et al., Science 269:1435-1439 (1995)), was tested. 5′NT-IgG-Au bound to the lung endothelial cell surface primarily in clusters on the plasmalemma proper that sometimes were at or near the caveolar diaphragm. 5′NT-IgG-Au did not traverse the endothelium to accumulate in the interstitium even after 15 min. Targeting lipid rafts under equivalent conditions did not result in rapid transcytosis. Thus, the TX3.833-Au transported to in the interstitium depended on targeting caveolae.

Sequential Transcytosis.

At 15 min with TX3.833-Au, it was also also found, where the attenuated endothelium was in close apposition to the alveolar epithelium, that some of the gold particles accumulating in the subendothelial space percolated through the basement membrane were taken up by epithelial caveolae for transport into and even across the cell. Gold particles were sometimes in endosomal-like structures and exiting caveolae opening into the air space. Although surprising because little TX3.833 antigen was detected in the epithelium by immunogold EM, it is possible that where the interstitial space separating the endothelium from the epithelium is small enough to allow higher concentration of transcytosed gold particles, the epithelial caveolae could take up and transport the gold particles by fluid-phase mechanisms. Perhaps more likely, the TX3.833 antigen is expressed at lower levels in the epithelial caveolae. Thus, transcellular transport by caveolae occurs not only in endothelium but also in epithelium and this sequential transcytosis can be used to overcome rapidly both cell barriers.

TX3.833 Targets Dynamic Caveolae.

Some caveolae maybe static (Severs, N. J., J. Cell Sci 90:341-8 (1988); Rippe, b. and Haraldsson, B., Acta Physiol. Scand 131:411-428 (1987); Bundgaard, M. et al., Proc. Natl. Acad. Sci. USA 76:6439-6441 (1979); Bundgaard, M., Federation Proc. 42:2425-2430 (1983)). To address whether TX3.833 targets dynamic caveolae capable of budding, a reconstituted budding assay (Oh, P. et al., J. Cell Biol. 141:101-114 (1998); Schnitzer, J. E. et al., Science 274:239-242 (1996)) was performed on P from lungs perfused with TX3.833. GTP induced plasmalemmal budding of caveolae that were collected as free floating vesicles containing the injected TX3.833 as well as caveolin-1 and TX3.833 antigen but not β-actin. This budding required GTP with active dynamin and was inhibited by nonhydrolyzable GTPγS and K44A mutant dynamin (Oh, P. et al., supra). Thus, TX3.833 can indeed target dynamic caveolae that require GTP hydrolysis by dynamin for their fission from the plasma membrane to form free transport vesicles.

Tracking Caveolae Targeting and Transcytosis in vivo.

Proteins and possibly other elements in the circulating blood increase the restrictiveness of the endothelial cell glycocalyx and can prevent access to caveolae (Schneeberger, E. E. and Hamelin, M., Am. J. Physiol. 247:H206-H217 (1984)). To test the ability of TX3.833 to target lung caveolae in vivo, TX3.833-Au was injected into rat tail veins and 15 min later processed the lung tissue for EM. TX3.833-Au could target the lung endothelial caveolae rather selectively. Gold label could be detected in luminal, abluminal, and apparently cytoplasmic caveolae. Transcytosis was observed with gold particles seen exiting abluminal caveolae to the underlying subendothelial space under normal physiological conditions in vivo. This visualization of caveolae targeting and transcytosis in vivo was consistent with the antibody-specific targeting of drug described herein as well as the lung subfractionation analysis showing ¹²⁵I-TX3.833 (10 min after injection) to be enriched in V at levels 100-fold>¹²⁵I-contol IgG (data not shown).

Lung-Specific Delivery and Bioefficacy of TX3.833-Drug Conjugates.

An antibody targeting lung caveolae in vivo could be useful as a carrier to achieve tissue-specific drug delivery. To test TX3.833 as a targeting vector, it was conjugated to various drugs and examined in vivo delivery of the immunoconjugate relative to the native drug. All TX3.833-drug conjugates showed greatly increased lung targeting up to 172-fold greater than drug alone (Supplemental Table 1).

TABLE 2
Immunoconjugation of Various Drugs
Increases Delivery to Rat Lung

| | % inj/g | Enhanced | % in |
|---|---|---|---|
| DRUG (IV) | tissue | delivery | lung |
| ³H-Daun | 0.63 | | 0.65 |
| TX3.833-³H-Daun | 17 | 27 | 18 |
| ¹²⁵Iodide | 0.39 | | 0.45 |
| ¹²⁵I-TX3.833 | 67 | 172 | 75 |
| ¹²⁵I-dgRA | 0.30 | | 0.32 |
| TX3.833-¹²⁵I-dgRA | 15 | 50 | 15 |

Each value represents the mean (N≧2). Thirty minutes after IV injection, lung uptake was determined as the percent of the original injected dose per gram of tissue (% inj/g) for the native or conjugated radiolabeled drug. The enhanced drug delivery is expressed as the antibody-drug conjugate relative to the native drug. The “% in lung” designates the total drug in the lung as a percentage of the original dose. Conjugation of ³H-daunomycin (“Daun;” DuPont NEN, Boston, Mass.) to TX3.833 was performed as described (Hurwitz, E. et al. (1975) Cancer Res. 35, 1175-1181). Other conjugations described in Methods. The conjugation procedure which minimally modifies proteins (Cumber, A. J. et al., Methods in Enzymology 112:207-225 (1998)), namely conjugation to radio-iodine, gave the greatest degree of targeting (172-fold>iodine alone). The other conjugates (the procedures that do compromise antibody-antigen binding (Oh, P. and Schnitzer, J. E., in Cell Biology: A Laboratory Handbook, ed. Celis, J. (Academic Press, Orlando), Vol. 2, pp. 34-36 (1998)) provided 27-50-fold greater lung delivery. The biodistribution analyses showed that the TX3.833 antibody did indeed target various drugs specifically to the lungs.

The bioefficacy of targeted drug was examined using dgRA immunotoxin which is highly toxic in small amounts but requires internalization by cells for A chain release into the cytoplasm and subsequent cell death (McIntosh, D. P. et al, FEBS Lett. 164:17-20 (1983)). At 6 hours and more so at 24 hours after IV injection, the TX3.833-dgRA-treated rats showed acute respiratory distress and general malaise (very rapid, shallow breathing; keeping stationary and focused on just breathing). Histological tissue examination, revealed lung tissue disruption with edema, blood infiltration, and thickening of septa but no detectable damage to any other. EM also revealed a loss of endothelial junctional integrity, marked membrane vesiculation of both endothelial and epithelial cells, and the presence of surfactant bodies in the alveolar spaces. The rats treated for 24 h with controls (equivalent levels of control IgG-dgRA, unconjugated TX3.833, native dgRA alone or TX3.833 unconjugated but together with dgRA) appeared clinically and histologically normal. The damage to lung endothelial and epithelial cells is consistent with endothelial transcytosis of TX3.833-Au and uptake by underlying tissue cells. Thus, the cumulative data indicate that directing a drug to endothelial caveolae can provide tissue-specific targeting, transcytosis for access to cells inside the tissue, and localized bioefficacy in vivo.

Discussion

The concept of vascular targeting has evolved in the last 20 years from the failure of many directed therapies to reach their intended target tissue cells (Tomlinson, E., Advanced Drug Delivery Reviews 1:87-198 (1987); Schnitzer, J. E., N. Engl. J. Med. 339:472-4 (1998); Schnitzer, J. E., Trends in Cardiovasc. Med. 3:124-130 (1993); Denekamp, J., Progr. Appli. Microcirc. 4:28-38 (1984); Burrows, F. J. and Thorpe, P. E., Pharmacol. Ther. 64:155-174 (1994)). Targeting endothelium because of its inherent IV accessibility has potential but so far requires key “proof of principle” in vivo. Although many attempts have been made to identify tissue-specific targets on vascular endothelium and to develop tissue-specific probes for vascular targeting (Hughes, B. J. et al., Cancer Res. 39:6214-6220 (1989); Pasqualini, R. and Ruoslahti, E., Nature 380:364-366 (1996)), directed delivery in vivo has not met theoretical expectations. A lung-targeting monoclonal antibody has been reported but the antigen is thrombomodulin which is expressed by many cells, including endothelia of several organs (Hughes, B. J. et al., supra). Immunotargeting the pan-endothelial marker, PECAM, can improve delivery to the lung (5-fold over control IgG) but only when the antibody is biotinylated and complexed with streptavidin (36). Screening phage display libraries in vivo for tissue-homing peptides has provided modest increased tissue delivery (Arap, W. et al., Science 279:377-380 (1998); Pasqualini, R. and Ruoslahti, E., Nature 380:364-366 (1996); Rajotte, D. et al., J. Clin. Invest. 102:430-7 (1998)) with <1% of the injected dose and relative targeting indices of 2-35-fold more than control phage and 3-80-fold more delivery than to the brain (note the well-known blood brain barrier has, as expected, the least non-specific binding and uptake). Using this last criteria, TX3.833 targeting index is >1000-fold more to lung than brain. TX3.833 as a probe has the specificity and affinity as well as the tissue- and cell-selectivity to validate, for the first time, the vascular targeting strategy by achieving theoretical expectations with high-level tissue targeting in vivo. Perhaps more importantly, it targets dynamic caveolae to overcome the endothelial cell barrier for access to underlying tissue cells.

Transport Pathway and Mechanism.

The cumulative in vivo and in situ data show that: i) caveolae can contain a tissue-specific endothelial protein, ii) an antibody can selectively and rapidly target and enter caveolae of microvascular endothelium in a specific tissue, and iii) targeting caveolae greatly increases the transendothelial transport and tissue accumulation over control antibodies (TTI≧150). Little transport or tissue accumulation is observed with physically similar, isotype-matched control antibodies that differ from TX3.833 in their ability to recognize a specific caveolar antigen. Thus, it is the specific entry and binding within the caveolae, and not just binding to the endothelial cell surface or another microdomain, such as lipid rafts, nor fluid-phase uptake by the caveolae that mediate the rapid and selective transendothelial transport of TX3.833-Au. The graded, time-dependent movement of the caveolae-targeting immunoprobes across the cell barrier cannot be explained by a nonspecific transport pathway (i.e. intercellular junctions) and back-filling of the abluminal aspect of a static, branched caveolar system, as logically proposed previously (Bundgaard, M., Federation Proc. 42:2425-2430 (1983)). It is now clear from appropriate controls that caveolae can mediate selective transendothelial transport, although the exact mechanism for this transcytosis requires further elucidation.

Application for Site-Directed Pharmacodelivery.

A selective caveolae targeting strategy may be useful for directed therapeutic delivery for the treatment of many diseases. By targeting caveolae at the luminal surface of endothelium in a single tissue, where the caveolae can transport the antibody from the blood directly into the target tissue, it was found that antibodies can indeed provide site-directed delivery in vivo with tissue accumulations reaching as high as 89% in just 30 min. Much of this lung-specific accumulation is antibody that has crossed the lung endothelial cell barrier by caveolae-targeted transport to become available for uptake by underlying tissue cells. Here, this process has been visualized by EM to show TX3.833 directing gold particles to the lung caveolae for rapid transendothelial transport from the circulating blood into the tissue. Like many drugs and gene vectors, the gold particles, alone or conjugated to control antibodies, do not readily cross the endothelial cell barrier. When conjugated to potential drugs or toxins, drug delivery by TX3.833 was selective to the lung and enhanced by up to 172-fold. TX3.833-dgRA conjugates selectively damaged the lung, primarily through the destruction of the alveolar endothelial and epithelial cells. Thus, an antibody targeting caveolae can be a carrier to provide tissue-specific pharmacodelivery and bioefficacy through overcoming the endothelial cell barrier that normally restricts delivery to the underlying cells of the tissue.

In the quest to study the basic transport function of caveolae in endothelium, the data presented herein demonstrate that caveolae can indeed transcytose select cargo in endothelium as well as epithelium. A lung-, microvessel-, and caveolae-specific monoclonal antibody which targets the lung in vivo has been produced. After intravascular administration, TX3.833 rapidly targets dynamic caveolae of the lung microvascular endothelium and is rapidly transported across the cell for release to the interstitial space where it can be taken up by the caveolae of underlying epithelial cells and transported to the alveolar space. As such, caveolae may provide a selective and useful vesicular trafficking pathway not only to endocytose or transcytose select endogenous molecules but also to overcome the once seemingly insurmountable cell barriers to effective site-directed therapeutic delivery in vivo. Moreover, proteomic analysis of endothelial cell plasma membranes and their caveolae reveals several tissue-specific proteins that are differentially expressed in normal organs and in various solid tumors (unpublished data). Thus, a strategy of targeting caveolae of endothelium and epithelium offers exciting possibilities for achieving site-directed drug- and gene therapy of various diseases in vivo.

While this invention has been particularly shown and described with references to preferred embodiments thereof, it will be understood by those skilled in the art that various changes in form and details may be made therein without departing from the scope of the invention encompassed by the appended claims. The teachings of all references cited herein are incorporated by reference in their entirety.

What is claimed is:

1. A method of delivering an agent of interest to a luminal surface of vascular endothelium in a tissue-specific manner, comprising the steps of:
a) selecting an agent of interest that binds to and localizes to a component of a microdomain of the luminal surface of the vascular endothelium upon contact with the luminal surface, wherein the component of the microdomain to which the agent binds and localizes is tissue specific, and wherein the microdomain is selected from the group consisting of: caveolae, G domains, and caveolae associated with G domains; and
b) contacting the luminal surface of vasculature with the agent of interest, thereby delivering the agent to the luminal surface of the vascular endothelium in a tissue-specific manner.

2. A method of delivering an agent of interest into and/or across a luminal surface of vascular endothelium in a tissue-specific manner, comprising the steps of:
a) selecting an agent of interest that binds to and localizes to a component of a microdomain of the luminal surface of the vascular endothelium upon contact with the luminal surface, wherein the component of the microdomain to which the agent binds and localizes is tissue specific, and wherein the microdomain is selected from the group consisting of: caveolae, G domains, and caveolae associated with G domains; and
b) contacting the luminal surface of vasculature with the agent of interest, thereby delivering the agent into and/or across the luminal surface of the vascular endothelium in a tissue-specific manner.

3. A method of delivering an agent of interest across a luminal surface of vascular endothelium and from one side of an underlying cell to another side in a tissue-specific manner, comprising the steps of:
a) selecting an agent of interest that binds to and localizes to a component of a microdomain of the luminal surface of the vascular endothelium upon contact with the luminal surface, wherein the component of the microdomain to which the agent binds and localizes is tissue specific, and wherein the microdomain is selected from the group consisting of: caveolae, G domains, and caveolae associated with G domains; and
b) contacting the luminal surface of vasculature with the agent of interest, thereby delivering the agent across the luminal surface of the vascular endothelium and from one side of an underlying cell to another side in a tissue-specific manner.

4. The method of claim 3, wherein the agent of interest comprises an active agent component and a transport agent component, wherein the transport agent component binds to and localizes to a component of the microdomain of the luminal surface of the vascular endothelium.

5. The method of claim 4, wherein the tissue is malignant.

6. The method of claim 4, wherein the agent of interest comprises a nucleic acid.

7. The method of claim 4, wherein the agent of interest comprises an immunotoxin.

8. The method of claim 4, wherein the active agent component is selected from the group consisting of: an antibody, a nucleic acid, a drug, a toxin, and a diagnostic agent.

9. The method of claim 4, wherein the transport agent component binds to and localizes to a molecule present on the luminal surface of a microdomain of the luminal surface of the vascular endothelium.

10. The method of claim 4, wherein the active agent component and the transport agent component are the same component.

11. The method of claim 10, wherein the active agent component is selected from the group consisting of: an antibody, a drug, a toxin, and a diagnostic agent.

12. The method of claim 4, wherein the transport agent component is selected from the group consisting of: an antibody, a peptide, an inactivated virus, a receptor, a ligand and a nucleic acid.

13. The method of claim 12, wherein the transport agent component is an antibody.

14. The method of claim 4, wherein the tissue is selected from the group consisting of: vascular, pulmonary, cardiac, cerebral, nephric, hepatic, endocrinous and intestinal tissue.

15. The method of claim 14, wherein the vascular endothelium of a tissue is vascular endothelium of cardiac tissue, and wherein the agent of interest comprises a selective stimulant.

16. The method of claim 14, wherein the vascular endothelium of a tissue is vascular endothelium of a blood vessel, and wherein the agent of interest comprises an anticoagulant.

17. The method of claim 14, wherein the vascular endothelium of a tissue is vascular endothelium of a blood vessel, and wherein the agent of interest comprises a nucleic acid encoding an anticoagulant.
